(19) United States
(12) Patent Application Publication
(10) Pub. No.: US 2009/0264383 A1
(43) Pub. Date: Oct. 22, 2009

(54) Inhibitor of Adenylyl Cyclase for Treating a Disorder of the Circadian Rhythm

(75) Inventors: Michael Hastings, Cambridge (GB); Elizabeth Maywood, Cambridge (GB); John O'Neill, Cambridge (GB)

Correspondence Address: MCANDREWS HELD & MALLOY, LTD, 500 WEST MADISON STREET, SUITE 3400, CHICAGO, IL, 60661, US

(73) Assignee: Medical Research Council, London (GB)

(21) Appl. No.: 12/298,947

(22) PCT Filed: May 18, 2007
(86) PCT No.: PCT/GB07/01835

§ 371 Date: Oct. 29, 2008

(30) Foreign Application Priority Data
May 19, 2006 (GB) 0610059.8

(51) Int. Cl. A61K 31/70; A61K 31/52

(52) U.S. Cl. 514/46; 514/263.1

(57) ABSTRACT

The invention relates to use of a composition comprising an inhibitor of adenylyl cyclase in the elongation of circadian rhythm, a method of extending the period of circadian rhythm in a subject, said method comprising administering to said subject an inhibitor of adenylyl cyclase, and to adenylyl cyclase inhibitor for use in the treatment of a disorder of the circadian rhythm. Preferably the inhibitor is a P-site inhibitor, preferably 9-(tetrahydrofuryl)-adenine. The composition may further comprise a JNK inhibitor.

### FIELD OF THE INVENTION

The invention relates to manipulation of the circadian clock. In particular, the invention relates to slowing or delaying its period in the treatment of disorders of the circadian clock.

### BACKGROUND TO THE INVENTION

Biological circadian clocks oscillate with an approximately 24-hour period. They are found in the majority of eukaryotes, as well as many bacteria. The circadian clock affects many aspects of behaviour and physiology, causing regular measurable variations in activity over the 24-hour period. The circadian rhythms of sleep, melatonin, secretion and body core temperature are generated by the suprachiasmatic nucleus of the hypothalamus, the anatomic locus of the mammalian circadian clock. Irregularities in the circadian clock underlie a range of clinical disorders.

Nearly 20% of employees in industrialized countries are employed, in shift work, which requires them to drastically change their sleep habits weekly or even daily. Increasing numbers of employees are needed to work unconventional shifts. Shift workers having difficulty sleeping during the day also have difficulty staying awake at work. The more fatigued they become, the more likely they are to experience “microsleep,” an involuntary bout of sleep brought on by sleep deprivation that lasts for a few seconds. Approximately 20% of shift workers report falling asleep during work, which increases the risk of industrial accidents and decreases productivity.

There are two main types of shift work. Employees may either work an unconventional nonfluctuating shift e.g. 11 p.m. to 7 a.m., or rotate between three different shifts covering the full 24 hour period. Both types of pattern can cause sleep disorders and related problems affecting circadian rhythm. Shift work change (SWC, also called ‘shift lag’ or ‘shift work sleep disorder’), is caused by a failure to adapt circadian rhythms to unconventional and/or changing patterns of sleep/wake behaviour demanded by shift working.

Treatment for this condition is limited. Behavioral and/or pharmacological remedies known in the art can only help to alleviate symptoms. One view in the art is that the body may never fully adapt to shift work, especially for those who switch to a normal weekend sleep schedule.

Delayed sleep phase syndrome (DSPS), also called phase lag syndrome, is a circadian rhythm sleep disorder. However, unlike jet lag or shift lag, delayed sleep phase syndrome is a persistent condition. In clinical settings, it is one of the most common complications of sleep-wake patterns.

Delayed sleep phase syndrome results from a desynchronization between the patient's internal biological clock and the external environment. In contrast to jet lag, this resynchronization is not activated by travel or change in external environment. Rather, the patient's propensity to fall asleep is delayed in relation to that of normal subjects.

DSPS patients are typically unable to fall asleep before 2 a.m. and have extreme difficulty waking early (e.g. by 7 a.m.). The main difficulty for patients with DSPS is functioning early in the morning. A Person with DSPS, often fails academically and/or struggles to retain employment, which can be socially damaging and often compromises their health in a recent study involving 5,000 participants, DSPS accounted for about 40% of disorders involving sleep-wake schedules.

Familial Advanced Sleep Phase Syndrome (FASPS) is characterized by very early sleep onset and offset. This disorder is especially prevalent in young adults. Profound phase advance of the sleep-wake, melatonin and temperature rhythms are characteristic of this disorder. The trait segregates as an autosomal dominant with high penetrance. There are no effective treatments for this disorder in the art.

Jet lag is detrimental to individuals' health and performance. There are economic problems associated with jet lag such as ineffectiveness and/or low productivity in the workplace. There are also hazards presenting an acute risk such as increased chances of accidents due to fatigue when operating machinery, driving or similar activities. Currently the only treatment for jet lag is melatonin therapy. This can produce a minor phase resetting effect on the circadian clock of approximately 30 minutes/day when administered in the evening. Melatonin treatment suffers from problems such as having a narrow temporal therapeutic window, and poor efficacy in humans. Furthermore, melatonin facilitates sleep and is therefore unsuitable when a period of alertness or attentiveness is required, for example driving away from an airport on arrival. Thus, there is a need for improved or alternative therapies to jet lag and related disorders.

It should be noted that the circadian clock is so robust that even temperature perturbations do not affect it. According to ordinary biophysical predictions, 5° C. reduction in temperature would be expected to produce a 34 hour clock period. However, despite a slight dampening the suprachiasmatic nucleus maintains a rhythm of very nearly 24 hours before, during and after treatment. The extreme stability and resistance of the clock to external factors presents a problem in its manipulation.

Fluctuations in cAMP levels have been observed throughout the daily cycle. cAMP has been regarded as a downstream effector whose levels may be controlled by the circadian clock. When cAMP has been studied in the prior art, it has been via acute manipulations. Typically, agonists or antagonists of the system have been applied and the acute effect monitored over a maximum of 24 or 48 hours. It has been suggested that cAMP analogues can produce a phase shift effect in the circadian rhythm. There has been no suggestion that any effect on the period of oscillation of the rhythm can be achieved. The view in the art is that cAMP operates at a level well below that of the circadian clock mechanism.

Marks and Birabil (2000 Neuroscience Volume 98 pages 311-315) disclose that infusion of adenylyl cyclase inhibitor into the rat central nervous system can enhance rapid eye movement sleep. Specifically, introduction of this compound into particular parts of the rat brain can produce long lasting increases in the time spent in REM sleep. There is no mention of circadian rhythm manipulation in this document. It should be noted that the induction and determination of REM sleep patterns is accomplished by a mechanism separate from the circadian rhythm. The frequency and period of REM in sleep is governed by localised brain waves and electrical pulses during the sleep cycle. Thus, there is no teaching regarding periodicity or the biological clock in Marks and Birabil.

The present invention seeks to overcome problem(s) associated with the prior art.

### SUMMARY OF THE INVENTION

cAMP is an important second messenger in all cells. Daily cycles in cAMP levels can be observed. These cyclical variations in cAMP concentration were thought to be an output from the circadian clock. However, it has been surprisingly shown by the inventors for the first time that cAMP is actually an intrinsic or structural part of the circadian clock. The invention is based on this remarkable discovery.

The difference between a structural feature of the circadian clock and a mere output is profound. There are numerous effectors of the circadian rhythm, and very many biological events can be seen to fluctuate or vary regularly throughout the 24 hour day. However, studying the various outputs or effectors does not permit the manipulation or adjustment of the fundamental underlying clock mechanism. The manipulation of the actual biological clock mechanism is rendered possible for the first time by the present invention. An appreciation of the significance of this work can be facilitated by comparison to a mechanical clock. If an operator manipulates the hands of a mechanical clock, they would appear to change the output. However, it will be apparent that they have in no way affected the mechanism of the clock, and the time keeping properties of that clock have not been altered. This represents the state of the art—at best, crude phase changes or gross alterations to effectors of the clock can be made. By contrast, according to the present invention the actual time keeping mechanism, i.e. the period of the circadian clock can now be manipulated. This is the first time that this has been possible.

The present inventors disclose that cAMP is at the heart of the circadian clock. By manipulating cAMP levels, it is possible to change the rate at which this clock keeps time i.e. the period of the clock can be manipulated.

Thus, in one aspect the invention provides use of a composition comprising an inhibitor of adenylyl cyclase in the elongation of circadian rhythm.

Preferably said inhibitor of adenylyl cyclase is a purine site (p-site) inhibitor.

In another aspect, the invention provides a method of extending the period of circadian rhythm in a subject, said method comprising administering to said subject a composition comprising an inhibitor of adenylyl cyclase. The inhibitor is administered in an amount effective to extend the period of the rhythm by the chosen amount. Guidance regarding exemplary doses is presented below.

Elongation has its normal meaning in the art and refers to extension or prolongation. Preferably elongation means to increase in time, i.e. to make longer. In the context of the present invention this means to increase the period of the circadian rhythm.

In another aspect, the invention provides use of a composition comprising an inhibitor of adenylyl cyclase for the manufacture of a medicament for a disorder of the circadian rhythm.

In another aspect, the invention provides use of a composition comprising an inhibitor of adenylyl cyclase for the manufacture of a medicament for jet lag.

In another aspect, the invention provides use of a composition comprising an inhibitor of adenylyl cyclase for the manufacture of a medicament for familial advanced sleep phase syndrome.

In another aspect, the invention provides rise of a composition comprising an inhibitor of adenylyl cyclase for the manufacture of a medicament for shift lag.

Preferably said inhibitor is a purine site ligand.

Preferably said inhibitor is specific for adenylyl cyclases.

Preferably said inhibitor does not act at the Gs_{α}-binding-site of adenylyl cyclase.

Preferably said inhibitor acts at the P-site of adenylyl cyclase.

Preferably said inhibitor is selected from the group consisting of 9-(tetrahydrofuryl)-adenine, 9-(cyclopentyl)-adenine and 2′,5′-dideoxyadenosine.

Preferably the inhibitor is 9-(tetrahydrofuryl)-adenine (SQ 22,536 or THFA) or 2′,5′-dideoxyadenosine.

Preferably said inhibitor is 9-(tetrahydrofuryl)-adenine.

The composition may consist of the inhibitor of adenylyl cyclase.

In another aspect, the invention provides an adenylyl cyclase inhibitor for use in the treatment of a disorder of the circadian rhythm.

Preferably said disorder is jet lag.

Preferably said disorder is shift lag.

Preferably said disorder is filial advanced sleep phase syndrome.

Preferably said inhibitor is a purine site ligand.

Preferably said inhibitor is specific for adenylyl cyclases.

Preferably said inhibitor acts at the P-site of adenylyl cyclase.

Preferably said inhibitor is selected from the group consisting of 9-(tetrahydrofuryl)-adenine, 9-(cyclopentyl)adenine and 2′,5′-dideoxyadenosine.

Preferably said inhibitor is 9-(tetrahydrofuryl)-adenine.

In another aspect, the invention provides a use or method as described above wherein said composition further comprises a c-Jun N-terminal kinase (JNK) inhibitor.

Suitably said JNK inhibitor is SP600125 (anthra[1,9-cd]pyrazol-6(2H)-one; 1,9-pyrazoloanthrone; SAPK inhibitor II).

In another aspect, the invention provides a composition comprising an adenylyl cyclase inhibitor as defined above and a JNK inhibitor.

In another aspect, the invention provides a composition as described above for use in medicine.

In another aspect, the invention provides a pharmaceutical pack or kit comprising
(i) an adenylyl cyclase inhibitor, and
(ii) a JNK inhibitor.

Suitably said adenylyl cyclase inhibitor is an adenylyl cyclase inhibitor as defined above.

Suitably said JNK inhibitor is SP600125 (anthra[1,9-cd]pyrazol-6(2H)-one; 1,9-pyrazoloanthrone; SAPK inhibitor II).

### Adenylyl Cyclase Inhibitors

Adenylyl cyclase (sometimes referred to as adenylate cyclase) is a family of proteins. There are numerous different isotypes. These isotypes can have differential tissue expression patterns.

There are numerous adenylyl cyclase inhibitors known in the art. Different classes of inhibitor can have different points of action. It is disclosed that G_{sα} acting inhibitors tend to reduce cAMP to zero, or to undetectable levels. The effect of this drastic reduction is ablation of the circadian rhythm. This leads to arrhythmic behaviour and loss of the clock function. Following removal of the inhibitor, clock function can be restored.

By contrast, purine-site (P-site) inhibitors reduce cAMP levels, but do not completely ablate them. P(purine)-site ligands inhibit via a mechanism preferably having at least one of the following features: non-competitive; dead-end; post-transition state mechanism; specific for adenylyl cyclases; preferably said inhibitors act via a mechanism having all of said features. According to the present invention, P site adenylyl cyclase inhibitors can be used to extend the clock period, i.e. to slow the clock mechanism. Advantageously, P site inhibitors do not completely remove clock function, but rather slow it in a controlled manner. Preferably the inhibitors is a P-site inhibitor. Preferably the inhibitor is not a Gsα-binding site inhibitor; preferably the inhibitor is not a GaS site inhibitor (e.g. MDL—MDL antagonises the action of forskolin (which acts at the Gsalpha site)). Preferably the inhibitor is not a Gi site inhibitor (e.g. pertussis toxin—Pertussis toxin (PTX) irreversibly ADP-ribosylates G_i and G_o proteins, inactivating them and preventing their inhibition of adenylyl cyclase (AC). PTX treatment affects amplitude and not period.).

P-site inhibitors and their properties are reviewed in Dessauer et al (1999 TIPS vol 20 p 205). ‘P-site ligand’ refers to a moiety which binds at the AC catalytic (purinergic) site. A ‘P-site inhibitor’ has this binding property and inhibits enzyme activity.

An important property of p-site inhibitors is that they are not competitive with respect to ATP (the enzymatic substrate). Non-competitive/uncompetitive inhibitors affect the reaction rate, by altering the stability of the enzyme substrate or enzyme-product complex. In the case of AC, its rate is determined by two factors: 1) the rate of the cyclisation reaction and 2) the rate of release of PPi (one of the products—inorganic phosphate). P-site inhibitors bind to the purinergic hydrophobic pocket (following dissociation of cyclic AMP) and stabilise the conformation of the post-transition state enzyme-product complex, thus slowing the release of PPi (see Dessauer et al ibid-incorporated herein by reference), and leading to an accumulation of the enzyme-PPi complex. Thus, p-site inhibitors form a dead-end complex by binding to the active site of adenylate cyclase in the presence of pyrophosphate.

Determination of p-site binding is straightforward enzyme kinetics and has been carried for numerous p-site inhibitors in the art, for example as in Dessauer and Gilman (1997-JBC vol 272 pp 27787-95).

The skilled operator can carry out this assay and decide whether a moiety binds the p-site of AC. In case any further guidance is needed, a dissociation constant (Kd) of <100 uM would imply specificity. However, clearly with inhibitors it may be more appropriate to assess Ki, or IC50 or EC50 depending on operator choice. In any case it should be noted that due to the mechanism by which p-site inhibitors associate with AC, it is expected to only observe binding in the presence of Pi (inorganic phosphate).

Regarding AC activity assay, and thus determination of inhibition, this is a standard biochemical assay and can be performed in a number of ways well known in the art. In case any guidance is needed, preferably the AC assay is carried out as described in Onda et al (2001 JBC vol 276 pp 47785-47793, in particular first para R-col page 47786 which is incorporated herein by reference). Activity/inhibition may be determined for any suitable moiety such as the whole adenylyl cyclase, or purified catalytic domain(s). Determination of inhibition may thus be made by the skilled worker. In case any further guidance is needed, for an in vitro assay it is expected that even weak inhibitors will have IC50<5 mM.

Lithium can elicit an increase in circadian period. However, a link to AC inhibition by lithium is unproven, and unlikely. Indeed, lithium is known in the art to be preferentially active against glycogen synthase kinase, inositol monophosphatase, inositol polyphosphate 1-phosphatase, glycogen synthase kinase-3, fructose 1,6-bisphosphatase, bisphosphate nucleotidase, and phosphoglucomutase (all EC50<2 mM). By contrast, lithium's physiological EC50 against adenylyl cyclase is reported to be about 20 mM (Goldberg et al, 1988, Am J Renal Physiol), which is a concentration verging on toxicity in humans. Study of a physiologically relevant role of lithium, both in vivo and in vitro, supports the prevailing idea in the art that any action of lithium on AC/cAMP is likely to be indirect. Moreover, lithium's possible pharmacological action on AC (in vitro) is mostly thought to be mediated by competing with Mg2+ ions at the catalytic site, which is a general feature of its mechanism of inhibition (as opposed to non-competitively with ATP, in the case of preferred inhibitor of the invention THFA). As a consequence, lithium is not viewed as a p-site inhibitor in the field, or indeed as being at all specific for AC; lithium's mode(s) of action is considered to be through competition with Mg2+. In addition, whilst 10 mM lithium-elicits an increase in SCN period in vitro of 2-3 hours, it does not affect circadian period in 3T3 fibroblasts. By contrast, treatment according to the present invention such as with THFA increases period in all tissues tested. Furthermore, it is widely believed in the art that the period effects of lithium are mediated through inhibition of GSK3beta (EC50=0.8 mM). Thus, lithium is not a p-site inhibitor of adenylyl cyclase. Preferably the adenylyl cyclase inhibitor of the invention is not lithium.

As disclosed herein, certain types of adenylyl cyclase inhibitor produce a ‘complete’ knock-down of cAMP levels. This is not desirable for most aspects of the invention since it may ablate clock function or lead to a complete resetting of the clock. Preferably the invention reduces adenylyl cyclase activity without completely removing it. In this way, the invention advantageously relates to extension of the period (rather than removal of clock function). An example of a ‘complete’ knock-down adenylyl cyclase inhibitor is MDL-12,330A. An example of a preferred inhibitor providing a beneficial reduction in adenylyl cyclase (but not a ‘complete’ knock-down) is THFA. In other words, the invention preferably produces a low steady state of adenylyl cyclase activity (as effected by THFA) rather than a ‘complete’ knock-down (as effected by MDL).

Thus, in a broad aspect, the invention relates to the use of an adenylyl cyclase inhibitor in a manipulation of the circadian clock. Preferably, the invention relates to use of an adenylyl cyclase inhibitor in extension of the period of the circadian clock.

Preferably the adenylyl cyclase inhibitor is not a G_{sα} site inhibitor.

Preferably the adenylyl cyclase inhibitor is a P site inhibitor.

Preferably the adenylyl cyclase inhibitor is 9-tetrahydrofuryl adenine (also referred to as ‘THFA’ or ‘SQ22,536’); 2′,5′-dideoxyadenosine, or 9-(cyclopentyl)-adenine.

These inhibitors such as p-site inhibitors are commercially available e.g. from Sigma-Aldrich (2′,5′-dd-Ado (2′,5′-Dideoxyadenosine—cat no: D 7408); 9-THF-Ade (9-(Tetrahydrofuryl)-adenine, SQ 22,536—cat. no: S-153); 9-CP-Ade (9-(Cyclopentyl)-adenine—cat.no: C 4479):

Preferably the inhibitor is membrane permeable. Preferred P-site inhibitors which are membrane-permeable are 9-(tetrahydrofuryl)-adenine (SQ 22,536 or THFA), 2′,5′-dideoxyadenosine, and 9-(cyclopentyl)-adenine. All three have the advantage of being water soluble up to at least 125 mM. Preferably the inhibitor is 9-(tetrahydrofuryl)-adenine (SQ 22,536 or THFA) or 2′,5′-dideoxyadenosine. Preferably the inhibitor is 9-(tetrahydrofuryl)-adenine (SQ 22,536 or THFA).

Of the P-site inhibitors, THFA is the most preferred. It has the advantage of being most readily water soluble. The other inhibitors are less soluble (for some embodiments involving 2′,5′-dideoxyadenosine it is preferred to prepare stocks in DMSO prior to dilution).

9-(cyclopentyl)-adenine advantageously has greater metabolic and chemical stability than THFA and is also water soluble. However, it may be less potent than THFA and thus the dosing may need to be correspondingly adjusted as taught herein.

Regarding adenylyl cyclase, this is an enzyme with many family members. Different adenylyl cyclase isoforms have different expression patterns. Without wishing to be bound by theory, the proposed mechanism is that AC turnover is reduced in all tissues leading to a global slowing down of the clock i.e. throughout the organism being treated. Given the presence of different isoforms with different tissue expression patterns, it may be that more widely expressed isoforms of AC are better targets relative to those with restricted tissue expression. It may even be the case that particular adenylyl cyclase isoform-s have a more prominent circadian role. Nevertheless, advantageously adenylyl cyclase inhibitors are used which have activity across the adenylyl cyclase isoforms, thereby alleviating the need to use cocktails of isoform-specific inhibitors. Preferably the inhibitor is a P-site inhibitor, preferably THFA.

### JNK Inhibitors

C-jun N-terminal kinase (INK) is an important mitogen activated protein kinase (MAP kinase) family member. JNK is sometimes referred to as stress-activated protein kinase (SAPK).

Strikingly, the circadian actions of cAMP disclosed herein are protein kinase A-independent, but are dependent on the Epac family of guanine-nucleotide exchange factors, signalling through Jun N-terminal kinases (JNK) to activate circadian gene expression. Combination inhibition, such as simultaneous inhibition of AC and JNK activities causes unprecedented lengthening of circadian period in both SCN slices and fibroblasts.

Thus, the present invention relates to combinations of the treatments disclosed herein with inhibition of JNK, such as by administration of inhibitors of JNK.

Advantageous synergistic effects (i.e. enhanced extension of the clock period) are, obtained with the combination of JNK inhibitors and adenylyl cyclase inhibitors.

Suitable JNK inhibitors include JIP-1; dicoumarol; JNK inhibitor I (L-form) (e.g. 1 uM); INK inhibitor II (SP600125) (e.g. 40-90 nM); JNK inhibitor III; JNK inhibitor V (e.g. 70-220 nM); NPPB (5-nitro-2-(3-phenylpropylamino) benzoic acid); DMAP (N6,N6-dimethyladenine; 6-dimethyladenopurine). These are preferably used according to the manufacturer's instructions except as otherwise indicated, and are commercially available e.g. from Calbiochem/Merck biosciences.

A preferred Jnk kinase inhibitor is SP600125 (anthra[1,9-cd]pyrazol-6(2H)-one; 1,9-pyrazoloanthrone; SAPK inhibitor II). A preferred concentration is 30-50 uM.

Further JNK inhibitors include:

AEG-33783 from AEgera. AEG-33783 is a small-molecule neurotrophin mimetic. A suitable dose is 10 mg/kg/day. AEG-33783 is suitably administered orally or intravenously.

JNT-401 from Celgene. JNK-401 is suitably administered at 50 mg/kg.

CEP-11004 from Cephalon. A suitable dose is 1-10 mg/kg.

CEP-1347 from Cephalon. A suitable dose is 1 mg/kg SubQ 24 hour treatment.

Semapimod from Cytokine PharmaSciences; A suitable dose is 8 or 25 mg/m(2) via the gut.

AM-111 from Xigen. A suitable dose is 0.4 mg/ml or 2 mg/ml of AM-111 in a 250 ul gel formulation. This has been administered for other applications by transtympanic injection into the most affected ear.

XG-102 from Xigen A suitable administration is Vehicle (PBS; 2 ul) or D-JNKI-1 solution (containing 15.7 ng D-JNKI-1; JNKI-1 peptides available from Alexis) by injection.

ER-181304 from Eisai. A suitable dose is 100 mg/kg po bid.

AS-602801 from Merck KGaA. A suitable dose is 30 mg/kg once-daily. Can be repeated for 12 days.

Other possible JNK inhibitors may include. JNK inhibitors, from Merck KGaA; PMI-002 from Phytomedics; JSP1 inhibitor from Ceptyr; MX6 from Incyte Corp; XG-101 from Xigen; CC-930 from Celgene; JNK 930 from Celgene; JNK 9359 from Celgene; or Dz-13 (from any suitable source) or perifosine from AEterna Zentaris/Keryx Biopharmaceuticals (Perifosine (D-21,266) is a phospholipid derivative of alkylphosphocholine; a suitable dose is 100 mg po daily.

### Applications

The invention finds application in familial advanced sleep phase syndrome (FASPS). Subjects suffering from FAPSS typically show a 21-22 hour rhythm. According to the present invention, this condition is treated by administration of an adenylyl cyclase inhibitor in order to slow their biological clock to a 24 hour rhythm. The dosage given is preferably titrated in order to provide the lowest dose in order to achieve an approximately two hour delay to their usual circadian period.

The invention finds application in the treatment of jet lag. Typically, a body can adjust approximately one hour per cycle. Therefore, if a subject is jet lagged by a time difference of approximately five hours, the expectation is that it would take approximately five days for their circadian rhythm to become properly adjusted to the new time zone in which they are placed. According to the present invention, a subject suffering from jet lag may be treated by administration of an adenylyl cyclase inhibitor. The dose of the inhibitor would be adjusted by the operator according to the length of delay needed in the circadian rhythm. For example, a subject suffering jet lag after flying from London to New York would typically need approximately five hours adjustment to their cycle. Thus, the dose should be adjusted in order to provide an approximately 29 hour cycle. A preferred dose for this 29 hour cycle is a final serum concentration of 0.5 to 1 mM THFA—(depending on the retention of THFA this may require 100 mg/kg intake for an adult human). Preferably a treatment is provided during the circadian cycle in which the flight is made. For larger adjustments, a dose may be provided in two consecutive cycles. For example, for a subject flying from London to Los Angeles, the time difference is greater than 6.5 hours (the approximate maximum adjustment provided by a single dose) and therefore such a subject would be in need of doses in two consecutive cycles. Preferably the first dose is given in the cycle in which the flight is taken.

Preferably the invention is applied to Westward flights. This is because the invention is preferably used in the delay or extension or a circadian rhythm, i.e. a lengthening of a day, which is the effect observed with Westward jet lag.

The invention may be applied for Eastward jet lag. In this embodiment, the dose should be adjusted taking into account the need to advance the clock. According to the present invention, such advances should be treated as multiple retardations (multiple lengthenings) of the circadian cycle so that a net effect produce can be equated to an advance in the cycle. For example, if a subject flies East and experiences a twelve hour shift in the day/night cycle, then preferably two doses of adenylyl cyclase inhibitor would be given in two consecutive cycles, producing approximately a six hour shift in each of those two cycles, amounting to a twelve hour shift in total thereby synchronising the subject's circadian rhythm with the local time. As will be appreciated in this example, rather than advancing the subject's circadian clock, it has been retarded in order to achieve the same effect i.e. synchronisation of their clock with the new local time. This principle should be applied when operating the present invention to “advance” a subject's clock. In particular, combination treatments such as adenylyl cyclase inhibitor given with JNK inhibitor, are particularly suitable for Eastward jet tag applications. This is because the synergistic extra lengthening of the period using such combinations can produce A effect which can be equated to the advance of the clock as explained above. For example, giving a dual treatment with adenylyl cyclase inhibitor and JNK inhibitor can extend the period to approximately 36 hours; this will have an effect comparable to a 12-hour advance and therefore advantageously reduces the number of single treatments (and cycles) which might otherwise be required to produce the same adjustment (e.g. by multiple smaller retardations).

The invention may be applied to the treatment of shift workers. For example, when a worker embarks upon a series of night shifts, their clock may be adjusted by administration of adenylyl cyclase inhibitor in advance of the first night shift. This will extend their circadian cycle so that they could delay their metabolism, sleep, and other cycles, until near the end of their shift. If their shift exceeds approximately 6.5 hours (approximately the largest change available in a single cycle) then the close can be divided over two or more consecutive cycles as appropriate to achieve the necessary change in rhythm. Alternatively, shift workers may take doses on an ad hoc basis in order to adjust their clock ahead of a changing shift pattern.

### Combinations

Lithium treatment has been shown to lengthen circadian period. Lithium treatment typically lengthens the period to approximately 26 hours in organotypic slices, and to approximately 25 hours in whole animals. Without wishing to be bound by theory, it is believed that adjustment of the clock using lithium operates via a different mechanism to adjustment of the clock using adenylyl cyclase inhibitors. Thus, in one embodiment, lithium treatment may be advantageously combined with treatment using adenylyl cyclase inhibitors to produce an additive effect, which may advantageously reduce the number of administrations required to achieve a certain magnitude of adjustment.

Lithium is preferably used at 10 mM, preferably 3 mM, preferably at established doses for clinical use of lithium in humans such as 0.8-1.2 mM final serum concentration.

Melatonin has been used in, the prior art in the adjustment of circadian rhythm. Melatonin typically produces an acute phase shift of the circadian rhythm. Thus, in one embodiment, the invention relates to the administration of melatonin together with an inhibitor of adenylyl cyclase. In this way, the rhythm may be advantageously phase shifted and delayed in combination in order to achieve the desired adjustment.

Melatonin is preferably used at maunfacturer's recommended doses. Preferably melatonin is used at a dose of about 0.5-5 mg/day (i.e. per cycle) for an average adult human.

Jun N-terminal kinase (JNK) inhibitors can be used to extend the period of the circadian rhythm. Indeed; it is disclosed herein for the first time that JNK inhibitors can act synergistically with adenylyl cyclase inhibitors according to the present invention to produce a still further enhanced extension to the period of the circadian rhythm. This is particularly useful in applications such as eastward jet lag.

In another embodiment, the invention relates to a triple combination of lithium, melatonin and an adenylyl cyclase inhibitor which advantageously maximises the size of the adjustment which can be made in any one circadian cycle.

### Administration

In principle, multiple small adjustments to circadian cycle are preferable to a single large adjustment. An advantage of making multiple smaller adjustments is to ease the impact on the patient of making a single large adjustment in a particular given cycle. Thus, if a six hour adjustment was required then preferably two three hour treatments would be provided in two consecutive cycles.

Preferably a single cycle is adjusted by 2-12 hours, preferably by 2-10 hours, preferably by 2-8 hours, preferably by 2-6 hours preferably by 2-4 hours, preferably by 3 hours per cycle, preferably by 2 hours per cycle.

In general, smaller doses are used for smaller changes in period, and vice versa. Highest shifts are observed in NIH 3T3 fibroblasts. These have a circadian period of about 21 hours. In 1.2 mM THFA they shift to about 30 hours. Thus the probable maximum period increase in these conditions is approximately 9 hours. The suprachiasmatic nucleus (as the master clock) has a longer intrinsic period of 24 hours, and so shifts by approximately 6 hours under similar conditions.

Preferably administration is oral administration.

### Dose Levels

Typically, a physician will determine the actual dosage which will be most suitable for an individual subject. The specific dose level and frequency of dosage for any particular patient may be varied and with depend upon a variety of factors including the activity of the specific compound employed, the metabolic stability and length of action of that compound the age, body weight, general health, sex, diet, mode and time of administration, rate of excretion, drug combination, the severity of the particular condition, and the individual undergoing therapy.

Preferably THFA is orally administered, preferably in water. For mice, the concentration at administration is 4 mM in water.

Alternatively THFA may be administered in tablet form, preferably tablet form is used for humans.

As with any dosing, regime, attention must be paid by the operator to choice of an appropriate dose of THFA for chronic intake, balancing the rate at which it is metabolised/excreted. Furthermore, attention must be paid to what time of day the drug is administered (e.g. as would be the case in acute treatment) in considering any effects on the response to/efficacy of the drug (in circadian terms, attention should be paid to the Phase Response Curve as necessary).

Depending upon the need, the agent may be administered at a dose of from 0.01 to 30 mg/kg body weight, such as from 0.1 to 10 mg/kg, more preferably from 0.1 to 1 mg/kg body weight.

For SQ22,536 (THFA), a typical dose is 50 mg/kg for an adult human.

For 2′,5′-dideoxyadenosine, a typical dose is 60 ng/kg for an adult human.

For 9-(cyclopentyl) adenine, a typical dose is 75 mg/kg for an adult human.

The dose may preferably be split into two or more applications for administration in two or more separate cycles in order to advantageously reduce the impact of large changes in rhythm produced over a single cycle.

Within a given cycle, preferably the dose is given in a single administration.

Preferably the dose is given orally or by injection, preferably orally.

Depending upon the time delay desired for a given cycle, the dose of adenylyl cyclase inhibitor is varied accordingly. For example, to achieve maximum delay (of approximately 6.5 to 9 hours, preferably 6.5 hours for a whole animal), a dose of approximately 100 mg/kg of THFA would be administered to an average adult male human. To achieve a delay of approximately 3 hours, the dose would be approximately 30 mg/kg.

Treatment with 0.3% lithium lengthens period by approximately 2%.

Treatment with 25% D₂O increases period by approximately 5%.

### Pharmaceutical Compositions

The present invention relates to compositions comprising an inhibitor of adenylyl cyclase, and to uses of those compositions. In some embodiments the invention relates to uses of the inhibitor of adenylyl cyclase itself.

The present invention also provides a pharmaceutical composition comprising a therapeutically effective amount of an adenylyl cyclase inhibitor of the present invention and a pharmaceutically acceptable carrier, diluent or excipient (including combinations thereof).

The pharmaceutical compositions may be for human or animal usage in human and veterinary medicine and will typically comprise any one or more of a pharmaceutically acceptable diluent, carrier, or, excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Where the agent is to be administered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the fold of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some embodiments, the agents and/or growth factors of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If the adenylyl cyclase inhibitor is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

In a preferred embodiment, the pharmaceutical of the present invention is administered orally. Hence, preferably the pharmaceutical is in a form that is suitable for oral delivery.

### Administration Techniques

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components of die present invention may be administered alone but will generally be administered as a pharmaceutical composition—e.g. when the components are is in admixture with a suitable pharmaceutical excipient, diluent or carrier selected with regard to the intended route of administration and standard pharmaceutical practice. For example, the components can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

It is to be understood that not all of the components of the pharmaceutical need be administered by the same route. Likewise, if the composition comprises more than one active component, then those components may be administered by different routes.

If a component of the present invention is administered parenterally, then examples of such administration include one or more of: intravenously, intra-arterially, intraperitoneally, intrathecally, intraventricularly, intraurethrally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parental formulations under sterile conditions is readily accomplished by standard pharmaceutical techniques well-known to those skilled in the art. As indicated, the component(s) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluroalkane such as 1,1,1,2-tetrafluoroethane (HFA 134A™) or 1,1,1,2,3,3,3-heptafluoropropane (HFA 227EA™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) of the present invention can be administered in the form of a suppository or pessary, or it may be applied topically in the form of a gel, hydrogel, lotion, solution, cream, ointment or dusting powder. The component(s) of the present invention may also be dermally or transdermally administered, for example, by the use of a skin patch. They may also be administered by the pulmonary or rectal routes. They may also be administered by the ocular route. For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it can be formulated as a suitable lotion or cream, suspended or dissolved in, for example, a mixture of one or more of the following; mineral oil, sorbitan monostearate, a polyethylene glycol, liquid paraffin, polysorbate 60 cetyl esters wax, cetearyl alcohol, 2-octyldodecanol, benzyl alcohol and water.

### Pharmaceutical Combinations

The agent of the present invention may be administered with one or more other pharmaceutically active substances. By way of example, the present invention covers the simultaneous, or sequential treatments with an adenylyl cyclase inhibitor according to the present invention and one or more steroids, analgesics, antivirals or other pharmaceutically active substance(s) such as lithium and/or melatonin.

In particular, the present invention relates to the simultaneous, or sequential treatments with an adenylyl cyclase inhibitor according to the present invention and one or more JNK inhibitors.

It will be understood that these regimes include the administration of the substances sequentially, simultaneously or together.

The therapy can include the treatment of one or more of those disorders mentioned herein, or related complaint.

### The Circadian Clock

Circadian rhythms are tractable and robust. They are known to regulate behaviour, physiology and metabolism such as serum cortisol levels, body temperature, sleep patterns and other biologically significant characteristics. Circadian rhythms are typically observed to be within a range 23.5-24.5 hours. The rhythms are manifest at the whole organism level, the tissue level and even at the cellular level.

Oscillation of the circadian clock (occasionally referred to as the ‘biological clock’) has two key properties. The first is the period. This is the peak to peak (or trough to trough) time taken per cycle. In other words, this is the time taken for one complete “revolution” of the clock. In normal subjects, this is typically 24 hours. The second feature of the oscillation is its amplitude. This refers to the maximum reach or maximum values of the peaks and troughs of the cycling of the clock. The amplitude and the period are separate features of the oscillations of the clock. The present invention is primarily concerned with manipulation of the circadian rhythm/clock, that it to say with manipulation of the period of the rhythm/clock. Extension or elongation of the period of the rhythm/clock may for simplicity be simply referred to as extension or elongation of the rhythm/clock.

The circadian clock was originally thought to reside entirely in the suprachiasmatic nucleus (SCN). Removal of the SCN resulted in arrhythmic animals. However, more recently it has been observed that peripheral tissues display their own intrinsic oscillators, although these are typically less robust and of a lower amplitude, and under overall control of the SCN. Furthermore, circadian rhythms of gene expression have been detected in most cell types with up to 10% of the genome beg affected. These phenomena are also thought to be under the direct or indirect control of the SCN in order to achieve synchrony.

The accepted genetic model of the operation of the circadian rhythm is outlined below. Briefly, an activating transcriptional complex (including factors such as clock and bmal1) drives the transcription of inhibitory proteins such as Per1/2/3, Cry1/2 during early circadian day. These proteins then require many hours before they can stably be imported into the nucleus. This is partially explained by the observation that cytosolic phosphorylation of these proteins (by kinases such as GSK-3 etc) drives ubiquitin-mediated degradation of the negative limb clock factors. However, they also appear to be stabilised by complex formation with/phosphorylation by other kinases which eventually licenses the nuclear entry of the inhibitory complexes, where they are able to repress their own transcription. By the time these complexes have been disassembled and the cycle can begin again almost exactly 24 hours have passed. This transcriptional/translational feedback loop has additional targets, so-called “output clock genes”, and indeed a large number of genes have a circadian pattern to their transcriptional or translational profile. Every organism needs to reset its internal clock from external cues each day. One of the signalling mechanisms for communicating phase changes may be through 2nd messengers such as cAMP and/or calcium. One potential effector is the presence of activatable CREs in several circadian gene promoters. It is interesting to note that, of the clock genes, the only mutants which completely lose rhythmicity are those for PER2 and BMAL1. These are therefore currently regarded as essential components of the clock in this model.

Moreover, studies in cyanobacteria show that recombinant circadian proteins can sustain stable circadian cycles of auto-phosphorylation in vitro, in the absence of transcription, adding further complexity and confusion to the prior art view.

### Second Messengers

Ca2+ levels in the SCN rise prior to increase in firing rate. CRE binding-protein (CREB) phosphorylation follows light pulses. Active cyclic AMP/Ca2+Response Elements (CREs) are found in several “clock gene” promoters (e.g. Per1/Per2/Dec1). Ca2+ chelation rapidly dampens rhythms. Interneuronal communication is essential for SCN activity. Biphasic circadian variation in cAMP concentration can be observed. Circadian expression of several adenylyl cyclase isoforms can be detected.

cAMP

It is disclosed herein for the first time that cAMP signalling constitutes a new level of circadian regulation.

Cyclic nucleotides have been extensively studied as second messengers of intracellular events initiated by activation of many types of hormone and neurotransmitter receptors. Receptors that stimulate the conversion of ATP to cyclic 3′,5′-adenosine monophosphate (cAMP) are associated with G proteins. Binding of the hormone or neurotransmitter to its membrane-bound receptor induces a conformational change in the receptor that leads to activation of the α-subunit of the G protein. The activated Gₛ subunit stimulates, while the Gᵢ subunit inhibits-adenylyl cyclase (AC), furthermore some AC isotypes are known to be additionally regulated by intracellular calcium and/or protein kinase C (PKC). Stimulation of AC catalyzes the conversion of cytoplasmic ATP to cAMP. cAMP activates cAMP-dependent protein kinases, and other effectors, including protein kinase A, (PKA). By catalyzing the phosphorylation (activation or deactivation) of intracellular enzymes, cAMP-dependent kinases elicit a wide array of metabolic and functional processes. Negative regulation can occur in the pathway when phosphodiesterases (PDEs) catalyze the hydrolysis of cAMP to adenosine-5′-monophosphate (5′-AMP). Several families of phosphodiesterases (PDE-I-VI) act as regulatory switches by catalyzing the degradation of cAMP to adenosine-5-monophosphate (5′-AMP). PDE II is a low affinity PDE that can cleave both cAMP and cGMP. The activity of PDE II is stimulated by cGMP. PDE III is a low affinity PDE that is inhibited by cGMP and is involved in the regulation of smooth muscle and cardiac contraction. PDE IV is highly selective for cAMP and is the high affinity PDE present in most cell types.

We disclose for the first time that cAMP signalling pathways constitute a core component of the mammalian circadian clockwork. First, they sustain tissue and cellular rhythimcity in the circadian pacemaker of the suprachiasmatic nucleus (SCN). Second, they mediate inter-cellular synchronisation between SCN neurons. Third, they determine the intrinsic period of circadian pacemaking both in vitro and in vivo. We demonstrate that pharmacological inhibition of adenylyl cyclase (AC) leads to dramatic lengthening of period in the SCN. These roles of cAMP signalling in circadian timekeeping are general, also being evident in peripheral mammalian tissues and cell lines. According to the invention, it is believed that daily activation of cAMP signalling sustains progression of the transcriptional cycle. These findings reveal a novel and unanticipated point of circadian regulation in mammals, qualitatively different from the prior published transcriptional feedback model. We disclose novel therapeutic targets for circadian dysfunction.

It is disclosed herein for the first time that circadian adenylyl cyclase activation is essential for setting a 24 hour period. Inhibition of adenylyl cyclase leads to unprecedented period lengthening to as much as 30 or 31 hours, or even more.

The view in the art regarding operation of circadian rhythms involves a gene expression/protein feedback system, with second messengers such as cAMP being mere effectors. However, it is surprisingly disclosed herein that in fact cAMP is an essential element of the circadian clock, governing the period. This surprising discovery has allowed the present invention to be based on the manipulation of cAMP levels in order to manipulate the period of the clock.

The definitive test whether a candidate entity is a structural part of the clock mechanism is to assess period (i.e. length of cycle of the clock) under different conditions for said entity. Merely assessing an output or effector function is less rigorous and is therefore less preferred. Preferably the effect of a particular intervention on period is determined as indicative of an effect on the actual circadian clock mechanism.

The fact that second messenger such as calcium levels rise before firing suggests regulated process, more than just output, and led to the inference that it is an essential feature, which is supported by EDTA/BAPTA analysis. Thus, it is disclosed that cAMP is involved in circadian rhythm regulation. This is in contrast to the prior art view of it as a (non-essential) resetting mechanism—it had not been considered before the present invention that cAMP is playing a central role in generating circadian rhythms.

### Further Applications

The invention may be applied in the modulation of effects of other drugs. For example, the liver follows a physiological cycle controlled by the circadian rhythm. The activities of the liver in the day are different to those in the night. As a consequence, particular therapeutic agents may be more rapidly or more slowly degraded by the liver following administration at different times during a circadian cycle. Thus, the invention finds application in the manipulation of the circadian period followed by administration of a drug. This advantageously provides the best efficacy for a given drug according to the point in a circadian cycle at which it is administered. Thus, the invention may be used to modulate the response to particular drugs by modulation of the circadian rhythm.

It is well-known in the art that there is a daily peak in blood pressure at the start of the day. This peak in blood pressure has been correlated with higher incidences of stroke, cerebral infarction or coronary attacks. Incidence of these conditions can be up to 30% higher around the time of the daily peak in blood pressure. Thus, it is desirable to target blood pressure controls to the early morning blood pressure peak in order to combat this effect. Thus, the invention finds application in manipulation of the circadian rhythm in order to modulate early morning peaks in blood pressure and thereby, reduce the risk of blood pressure induced disorders such as those above.

The invention finds application in space travel. The day length on Earth is 23 hours and 56 minutes. Day length on Mars is 24 hours and 37 minutes. Thus, astronauts travelling to Mars need to adjust their circadian rhythms in order to suit the increased Martian day length relative to the day length of Earth. Thus, in one aspect the invention relates to extension of day length to approximately 24 hours 37 minutes by administration of an appropriate dose of adenylyl cyclase inhibitor to a subject in need of same. Thurs, according to the present invention travellers to Mars can increase the length of their circadian period to match that of the local environs, thus avoiding adverse-effect(s) of trying to live an Earth length day, on an alien planet.

The invention also relates to increasing circadian period length. Dose dependant period length increase, preferably in all tissues, enables Sufferers of Familial Advanced Sleep Phase Syndrome can be treated, bringing their period to 24 hours (chronic treatment). Shift workers can reduce the unpleasant side-effects and long term adverse health consequences of keeping unusual hours by adjusting their circadian phase to match their work day requirements through acutely manipulating their circadian period length. Sufferers from “jet lag” can reduce the unpleasant side-effects and long term adverse health consequences of changing time zone by adjusting their circadian phase to match the time zone of their destination through acutely manipulating their circadian period length.

The invention also relates to treatment of Seasonal Affective Disorder (SAD). Dose dependant period length increase, preferably in all tissues, enables sufferers from SAD (or winter depression) to increase their physiologically perceived duration of day length, and thus alleviate the symptoms of SAD which accompany day length shortening at higher latitudes.

The invention also relates to treatment of sleeping disorders. Dose dependant period length increase, preferably in all tissues, enables sufferers from insomnia and/or narcolepsy to have a means of coping with their condition through manipulation of physiologically perceived daytime and/or night time onset.

The invention also relates to treatment of depression. Lithium has a characterised role in treating depression. Its major pharmacological effect is mediated by inhibition of Glycogen Synthase Kinase 3β, this is leads to an modest increase in circadian period (˜30 minutes in mouse behavioural rhythms, ˜2 hours in organotypic tissue extracts). Because the SCN (suprachiasmatic nucleus—the master clock in mammals) has extensive reciprocal connections with the 5-HT centre the Median Raphe, and 5-HT treatment of SCN slices (in vitro) leads to p shifts, treatment which also increases circadian period in the SCN may advantageously have an anti-depressant action.

The invention also relates to treatment of appetite control. cAMP is an important second messenger in signalling the fasting state intracellularly, in pharmacological interference with this signalling pathway, and advantageously may have an action in suppressing appetite, particularly as the SCN has extensive communication with the orexin neurons of the hypothalamus.

The invention also relates to modulation of action of other drugs/treatments. Many drugs and healthcare treatments have been shown to have variable efficacy depending on the time of circadian day/night at which they are administered. Manipulation of a patient's physiological day/night length may advantageously extend the therapeutic window for maximal efficacy and/or reduce toxic side effects of other drugs.

The invention also relates to treatment of disordered sleep patterns in neurodegenerative diseases. Patients with advanced Parkinson's, Alzheimer's etc have disordered sleep patterns which makes them difficult to care for without 24-hour supervision. Manipulation of physiologically perceived day/night length may reduce these symptoms.

The invention may also relate to lifestyle/performance enhancement applications. For example, an increase in physiological night duration through acute treatment could help alleviate conditions such as sleep deprivation. An increase in physiological day duration through acute treatment could increase period of alertness, athletic performance, or productivity.

In a preferred embodiment when the adenylyl cyclase inhibitor is THFA, the invention also embraces treatment of blood pressure problems such as high blood pressure. cAMP is a key regulator of excitation-contraction coupling in the heart; this role is mainly mediated by PKA, thus the use of a non-competitive P-site inhibitor may advantageously reduce high heart rates.

In a preferred embodiment when the adenylyl cyclase inhibitor is THFA, the invention also embraces treatment of specific tissues. THFA is a membrane-permeable inhibitor; as such it may diffuse into multiple tissues, preferably equally well regardless of tissue type. By the addition of certain moieties to the THFA molecule, it may be targeted to specific tissues. e.g. non-blood brain permeable moieties would affect peripheral tissues only.

In a preferred embodiment when the adenylyl cyclase inhibitor is THFA, the invention also embraces cancer treatment, where THFA may have a tumour suppressor action.

In a preferred embodiment when the adenylyl cyclase inhibitor is THFA, the invention also embraces stroke treatment. THFA may have an anti-apoptotic role, thus administration following the occurrence of a stroke may reduce brain damage.

Although Gsα site adenylyl cyclase inhibitors are not useful in extension of the period of circadian rhythm according to the present invention, they may advantageously be applied in other aspects as noted herein.

The invention will now be described by way of examples which are intended to be illustrative rather than limiting in nature. In the examples, reference is made to the following figures:

### BRIEF DESCRIPTION OF THE FIGURES

FIG. 1 shows Activity Traces of per1::luc animals

FIG. 2 shows a graph of rhythms in VPAC2−/− slices

FIG. 3 shows a diagram

FIG. 4 shows a graph and a bar chart

FIG. 5 shows a graph

FIG. 6 shows graphs, a bar, chart and a diagram.

FIG. 7 shows a graph

FIG. 8 shows graphs and bar charts

FIG. 9 shows graphs

FIG. 10 shows a dose response curve

FIG. 11 shows a dose response curve

FIG. 12 shows a graph

FIG. 13 shows a graph

FIG. 14 shows the structure of SQ 22536 (9-(Tetrahydro-2′-furyl)adenine (THFA))

FIG. 15 shows inhibition at the Gsα site of AC suppresses circadian gene expression and desynchronises SCN neurons

(a) Representative traces showing that 10 μM forskolin (red) but not vehicle (black) increases circadian transcriptional amplitude in organotypic SCN slices from mPer1::luc, Vip2r−/− mice.

(b) Group data (mean+SEM, n=3) showing enhanced circadian gene expression (expressed relative to initial amplitude) in mPer1::luc, Vip2r−/− SCN slices following 10 μM forskolin (treatment effect, p<0.01, 2-way ANOVA).

(c) Representative traces show reversible suppression of circadian protein expression following addition of MDL-12,330A to PER2-LUC SCN slices.

(d) Group data (mean+SEM, n>3) show reversible, dose-dependent dampening of circadian gene expression (expressed relative to initial amplitude) by MDL-12,330A in PER2-LUC SCN slices (0=black, 0.5 μM=green, 1.0 μM=red, 2.5 μM=orange, 5 μM=blue). A second wash was required for recovery after highest doses of MDL.

(e) CCD imaging reveals loss of circadian amplitude in SCN neurons from PER2::LUC slice treated with 2.5 μM MDL-12,330A. Photomicrographs illustrate distribution of cellular circadian PER2::LUC expression across SCN slice before (left) and during (right) treatment. V-3rd ventricle, bar 500 um. Graph presents traces from 20 representative cells before and during MDL addition. Inset presents same data with expanded ordinate. Note disorganization of cellular profiles with MDL. Data representative of 3 slices.

(f) CCD imaging reveals desynchronisation if circadian timing-in-SCN neurons from PER2::LUC slice treated with 1.0 μM MDL-12,330A for over 7 days Photomicrographs illustrate distribution of cellular circadian PER2::LUC expression across SCN slice before (left) and during (right) treatment. Raster plot shows PER2::LUC expression in SCN cells before (−6 to 0 days) and after 1 week (+7 to 12) of MDL. Note loss of synchrony with MDL. Data representative of 3 slices.

FIG. 16 shows inhibition of AC catalytic p-site extends circadian period in SCN, peripheral tissues and 3T3 fibroblasts

(a) Representative traces show that addition of THFA to mPer1::luc organotypic SCN slices causes reversible increase of circadian period. Vehicle=black, 0.5 mM TBFA=red, 2.0 mM=blue.

(b) Dose-dependent, saturable period lengthening in mPer1::luc SCN (solid line is sigmoidal curve fit to mean data, dotted line is 95% confidence limits, n>3). Red data point indicates periods after wash-out (mean+SEM).

(c) Extended circadian period in 20 representative SCN neurons from PER2::LUC slice imaged by CCD and treated with 1.0 mM THFA. Data representative of 3 slices.

(d) Reversible period lengthening in SCN mPer1:luc slices treated with 2′5′-dideoxyadenosine (red), 0.5 mM THFA (blue), and 9-cyclopentyl adenine (green) (mean+SEM, n>3, p<0.01, 2-way ANOVA).

(e) THFA significantly (*p<0.05 vs. pre-treatment) prolongs circadian period in Clock heterozygous and homozygous mutant SCN (mean+SEM, n>3).

(f) THFA (2 mM) lengthens period in organotypic cultures of peripheral tissues from mPER2::LUC mice (for all tissues: n≧4, drug effect p≦0.02).

(g) Representative traces from NIH 3T3 cells transfected with mBaml1::luc reporter show period lengthening to THFA. Black=vehicle; red=0.3 and blue=1.2 mM THFA)

(h) Dose-dependent saturable period lengthening in 3T3 cells by THFA (solid line is sigmoidal fit to mean data, dotted line is 95% confidence limits, n>3).

FIG. 17 shows intra-cerebral infusion of THFA lengthens circadian period in mice in vivo

(a) Representative double-plotted, actograms of (left) vehicle- and (right) THFA-treated mice entrained to a photoschedule of 12 hours light (shaded) and 12 hours dark and fitted with osmotic mini-pump and central cannula directed at SCN (asterisk) Mice were then released into continuous dim-red light and free-running period determined Note longer period in THFA-treated mice.

(b) Group data (mean+SEM) reveal significant (p<0.01, t-test) lengthening of free-running period in vivo by THFA compared to vehicle.

FIG. 18 shows that cAMP regulates circadian gene expression via EPAC signalling

(a) Representative traces reveal that suppressed circadian gene expression in presence of MDL-12,330A can be rescued in PER2-LUC SCN slices treated with Epac agonist (arrow, 100 μM).

(b) CCD imaging reveals acute activation and synchronisation of cellular circadian gene expression in SCN slices pre-treated with MDL and then given Epac agonist. Upper panel shows raster plots of 20 representative cells, presented as graphical plots in lower panel. Data representative of 3 slices.

(c) Representative traces show that over-expression of Epac inhibitor Rap1S17N (red-curve) dampens circadian gene expression rhythms in NIH 3T3 cells transfected with mBmal1::luc, both before and after a medium change (arrow). Over-expression of wild-type Rap (blue) or empty vector (black) has no effect.

(d) Representative traces show suppression of circadian gene expression in 3T3 cells-transfected with mBmal1::luc reporter and treated with interfering RNA to either Epac1 (blue) or Epac 2 (red) compared to empty vector (black).

(e) Joint inhibition (blue) of AC (THFA 1 mM, red) and JNK (SP600125 20 uM, green) activities dramatically lengthens circadian period in PER2::LUC SCN slices (representative slices).

(f) Group data (mean+SEM, n>3) reveal significant additive lengthening of circadian period in PER2::LUC SCN slices by joint inhibition of AC (THFA 1 mM, TH) and JNK (SP600125 20 uM, SP)** p<0.01 one-way ANOVA; n.s.=not significant).

FIG. 19 shows a schematic model of how the circadian clockwork is a product of interlinked AC-dependent signalling pathways and transcriptional feedback loops.

The canonical feedback oscillator of the clock involves auto-regulatory feedback loops (green) driven by periodic, alternating activation and inhibition at DNA regulatory sequences such as E-boxes and ROREs. Transcriptional output from the loops is translated into various extra-cellular signals that sustain circadian biology. The circadian transcriptional loops are sustained by adenylyl cyclase (AC) signalling, via Epac and JNK (red)-likely acting through AP-1 DNA regulatory sequences in clock genes. Circadian cycles of AC activity are a product of output from the intra-cellular transcriptional loops (e.g. cyclical expression of AC) and afferent stimuli acting upon Gsα and Gsi (blue). Interference with either the transcriptional feedback loop or AC signalling can either suspend the clockwork or alter its period. Within the circuitry of the SCN, two further special conditions apply. First, the extra-cellular output of one cell in the form of VIP neurosecretion will provide synchronising and sustaining cues to post-synaptic cells. Second, retinal innervation mediated by glutamatergic cues can synchronise clock cells by resetting the transcriptional feedback loop via [Ca++]i-dependent CRE DNA regulatory sequences. In tissues other than SCN, AC and [Ca++]i-dependent pathways will be addressed by alternative, tissue-specific synchronising actors, and likely involve extensive interaction between signalling cascades.

FIG. 20 shows differential effects of AC inhibitors MDL and THFA on circadian gene expression and cAMP levels.

(a) ELISA of cAMP in levees in control synchronised cultures NIH 3T3 fibroblasts reveals loss of measurable cAMP in presence of MDL-12,330A (mean+SEM, n=3 for all):

(b) Addition of 5 μM MDL-12,330A to wild-type mPer1::luc slices dampens transcriptional amplitude. Note prolonged delay until recovery of competent circadian transcription following repeated wash-out (arrow).

(c) Representative trace shows rapid decline in circadian gene expression in 3T3 cells transfected with mBmal1::luc reporter. Prolonged and repeated wash-out (arrow) was again necessary to restore rhythmicity.

(d) Group data reveal dose dependent suppression of circadian gene expression by MDL-12,330A in 3T3 cells transfected with mBmal1::luc reporter, and reversal by wash-out (mean+SEM, n≧3, drug effect p<0.0001, 2-way ANOVA).

(e) Addition of THFA to NIH 3T3 cells, suppresses peak levels but maintains basal levels of cAMP (mean+SEM, n=3 for all, control data as in (a)).

(f) Circadian gene expression patterns in fibroblast cultures parallel to those in (e) but transfected with mBmal1::luc show prolongation of circadian period is associated within loss of peak cAMP titres.

FIG. 21 shows inhibition of PKA does not affect circadian gene expression in SCN (a) Representative traces from mPER2::LUC SCN slices show that treatment with inhibitors against PKA regulatory-subunit (red trace, 300 μM Rp-8-Br-cAMPS+100 μM Rp-8-CPT-cAMPS) has no significant effect compared to vehicle (black trace) on circadian period nor transcriptional amplitude.

(b), (c) Group data for show no significant effect of the two PKAR inhibitors on transcriptional amplitude (n=4, p=0.89, 2-tailed t-test) nor circadian period (n=4, p=0.15, 2-tailed t-test)

(d) Representative traces from mPer1::luc SCN slices with inhibitors against PKA catalytic subunit (1 μM KT5720) show no significant effect on period nor transcriptional amplitude.

(e), (f) Group data show no significant effect of PKAC inhibitor on transcriptional amplitude (n=3, p=0.34, 2-way ANOVA interaction) nor circadian period (n=3, p=0.75, 2-way ANOVA interaction).

FIG. 22 shows Epac/JunK pathways mediate circadian effects of cAMP/AC signalling

(a) Group data reveal acute induction of circadian gene expression by Epac agonist in presence of MDL. Before treatment there was no significant difference between groups (p=0.8) but 100 μM Epac agonist significantly increased luciferase activity (p=0.016, drug effect 2-way ANOVA) (Mean+SEM, n=5).

(b) Group data (mean+SEM, n=4 per treatment) confirm significant (p<0.01) suppression of circadian amplitude in 3T3 cells following Epac inhibition by Rap1S17N.

(c) Group data (mean+SEM, n=5 per treatment) show reduced an amplitude of circadian gene expression in 3T3 cells transfected with in Bmal1::luc reporter and treated with interfering RNA to either Epac1 (blue) or Epac 2 (red) compared to empty vector (black) (drug effect p<0.01 by 2-way ANOVA).

(d) Representative western blot of 3T3 cell extracts (duplicate lanes) shows that addition of Epac agonist (100 μM, 2 hours) reverses the decline in JNK phosphorylation following 40 hours incubation with MDL (5.0 μm). Epac had no effect on CREB phosphorylation.

(e) Group data (mean+SEAM, n=4 independent experiments) reveal significant increase in JNK activation by Epac in 3T3 cells-treated with MDL (* p<0.05 one-way ANOVA).

(f) Representative traces show that simultaneous inhibition of both AC (THFA 1 mM) and JNK (SP600125 20 uM) activities dramatically lengthens circadian period in 3T3 cells (red trace) compared to vehicle treatment (black trace).

(g) Group data (mean+SEM, n<3) reveal significant additive lengthening of circadian period in 3T3 cells transfected with Bmal;;luc reporter by joint inhibition of AC (THFA 1 mM, TH) and JNK (SP600125 20 uM, SP) and non-additive effects of THFA and IBMX (300 uM, IB) (** p<0.01 by one-way ANOVA, n.s.=not significant).

### EXAMPLES

### General Methods

Studies were licensed under the UK Animals (Scientific Procedures) Act 1986, with Local Ethical Review by the Medical Research Council and the University of Cambridge. Per1::luciferase and; Per2-luc transgenic mice were used. For organotypic slice culture, brains were removed from pups 5-10 days old and sectioned at 300 um with a McIlwain “Tissue Chopper.” Slices were sorted and trimmed to contain principally SCN tissue and placed onto a Millipore membrane insert (PICMORG) for culture at 37° C. in 5% CO2 as described previously (Gainer et al, 1998). For long-term recordings, slices were transferred to 1.1 ml HEPES buffered medium with 100 uM beetle luciferin (Promega) in a glass-bottomed Petri dish sealed with a coverslip and vacuum grease. Total bioluminescence was recorded with Hamamatsu photomultiplier tube assemblies housed in a light-tight 37° C. incubator. Photomultiplier recordings were expressed as counts per second integrated over 6 min sample bins. Periods are peak-to-peak averages for not less than 3 cycles, with at least 3 replicates for each data point. cAMP assays were determined by R&D systems 3rd generation cAMP ELISA kit. All drugs were purchased from Sigma Aldrich, made up as a stock solution in medium or DMSO; then added to tissue medium.

### Example 1

### Cyclic AMP-Dependent Signals Sustain Molecular Time-Keeping in Mammals and Determine Circadian Period

### Introduction

Circadian timing in mammalian cells is based upon an auto-regulatory transcriptional/post-translational feedback loop, pivoted around the rhythmic expression of Period and Cryptochrome genes. Although circadian activation of various second-messenger signalling cascades (including cyclic nucleotides, MAPK and calcium) has been widely observed, their role within the clockwork has been viewed primarily in respect of entrainment, most obviously induction of Per expression. In VIP2 receptor knockout mice (Vip2r−/−), however, the molecular clockwork is suspended in most suprachiasmatic nucleus (SCN) neurons. This receptor signals via adenylyl cyclase (AC). We therefore sought to test the role of AC signalling in maintaining circadian time-keeping in mammals, using real-time bioluminescent recording of circadian gene expression.

### Experimental

Inhibition of the Gsα-binding site of AC caused a dose-dependent, reversible, dampening of circadian gene expression from Per1::luciferase organotypic SCN slice cultures, monitored by photomultiplier tubes. To determine the generality of this effect, we examined 3-T3 fibroblast cells. Abrogation of the endogenous oscillation of cAMP concentration stopped circadian gene expression as reported by Bmal1::luciferase reporter constructs P-site AC inhibition prolonged fibroblast period from ca. 21 hours to ca. 30 hours. Comparable treatment of Per1::luciferase SCN slices lengthened circadian period up to 30 hours.

Loss of cAMP signalling may account for the loss of molecular timekeeping in the SCN of the Vip2r−/− mutant mouse. More generally, cAMP signalling pathways are essential to sustain, mid regulate period of, the mammalian circadian clockwork, both in SCN and in peripheral cells. Thus it is demonstrated according to the present invention that P-site adenylyl cyclase inhibition prolongs the circadian period.

### Example 2

### Demonstration of Prolongation of Circadian Rhythm Using Adenylyl Cyclase Inhibitors

We disclose the cyclic AMP (cAMP) pathway as a new mode of manipulating circadian rhythms in mammals. We show that observed twice-daily, intracellular changes in cAMP levels are an essential feature of the clock, rather than an output. Our manipulation of cAMP synthesis confirms this role. Furthermore, the invention identifies previously characterised intracellular targets for this application.

Firstly, we, have found that treatment of a range of mouse tissues (e.g. organotypic brain and kidney slices, fibroblasts etc) with common, commercially available-inhibitors of the enzyme adenylyl cyclase-prolongs circadian period (as detected by a range of bioluminescent reporters) from ˜24 to >30 hours, without dampening them. This effect is unprecedented in circadian physiology/pharmacology.

The preferred inhibitors according to the present invention belong to a class historically called P(purine)-site ligands, which inhibit via a non-competitive, dead-end, post-transition state mechanism which makes them specific for adenylyl cyclases. The most effcacious of these inhibitors is THFA (9-(Tetrahydrofuryl)-adenine), or SQ 22,536].

THFA is membrane-permeable, water-soluble and has previously safely been used on live rodents via intracranial injection (e.g. Marks et al, Neuroscience, 2000 ibid.). Furthermore, we have not observed any toxic effects in vitro during chronic incubations with THFA for periods of up to 7 days, at concentrations as high as 2 mM. Period reverts to its normal value of approx. 24 hours soon after removal of the drug.

We have found that application of THFA to mouse fibroblasts reduces the detectable, endogenous, biphasic rise in cAMP, without affecting its nadir. Oral delivery of this drug to mice thus advantageously alters their circadian period without compromising basal cAMP functions, demonstrating the utility as treatment for disrupted, human circadian rhythms.

The invention finds application in targeting these pathways, particularly in circadian dysfunctions such as sleep disorders (insomnia and sleep phase), shift-work disturbances and jet-lag.

### Example 3

### Circadian Rhythm

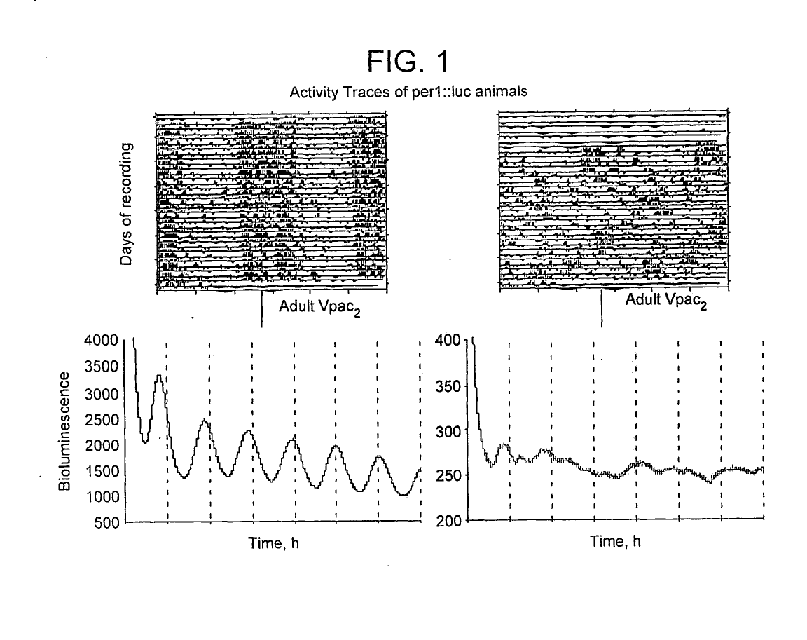

FIG. 1 shows activity traces of per1::luc animals with different genetic states with respect to VPAC2. Animals negative for the receptor are behaviorally disorganised.

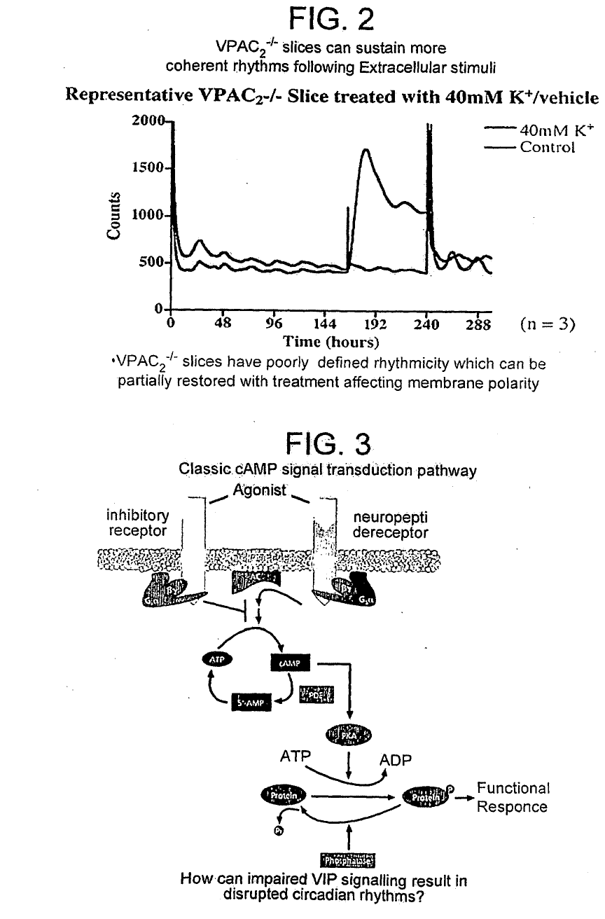

FIG. 2 shows VPAC2−/−-slices can sustain more coherent rhythms following extracellular stimuli.

FIG. 3 shows a diagram of a classical cAMP signal transduction pathway.

VPAC mice are poorly rhythmic in their behaviour. This is reflected in the poor rhythmicity and low amplitude SCN rhythms. Electrophysiological measurements have shown that an additional phenotype of VPAC SCN is that they are hyperpolarised. Thus, they can be “kick-started” by K+, or AP-4 (sodium channel blocker). This temporally restores synchrony and some amplitude to the slice.

This effect is likely mediated, at least partly by an extra-cellular calcium flux, as it can be abrogated by pretreatment of the slice with 1.6 uM EGTA.

However, VPAC receptor is not an ion channel, but is actually a GPCR which is understood to signal through Gs. We thus investigated matters further by studying cAMP.

Cyclic Nucleotide Metabolism—cAMP

Cyclic nucleotides have been extensively studied as second messengers of intracellular events initiated by activation of many types of hormone and neurotransmitter receptors. Receptors that stimulate the conversion of ATP to cyclic 3′,5′-adenosine monophosphate (cAMP) are associated with G proteins. Binding of the hormone or neurotransmitter to its membrane-bound receptor induces a conformational change in the receptor that leads to activation of the a-subunit of the G protein. The activated Gs subunit stimulates, while the Gi subunit inhibits adenylyl cyclase (AC). Stimulation of AC catalyzes the conversion of cytoplasmic ATP to CAMP.

Cummings et al had suggested an essential role for cAMP (in a biochemical model for circadian clocks) in 1975, but this idea was subsequently disproved in Neurospora.

The current view in the alt is that cAMP involvement is limited to a role in phase resetting.

### Example 4

### Role of Adenylyl Cyclase in Circadian Rhythm

### Adenylyl Cyclase Agonist

Forskolin is an adenylyl cyclase agonist with a well-characterised action.

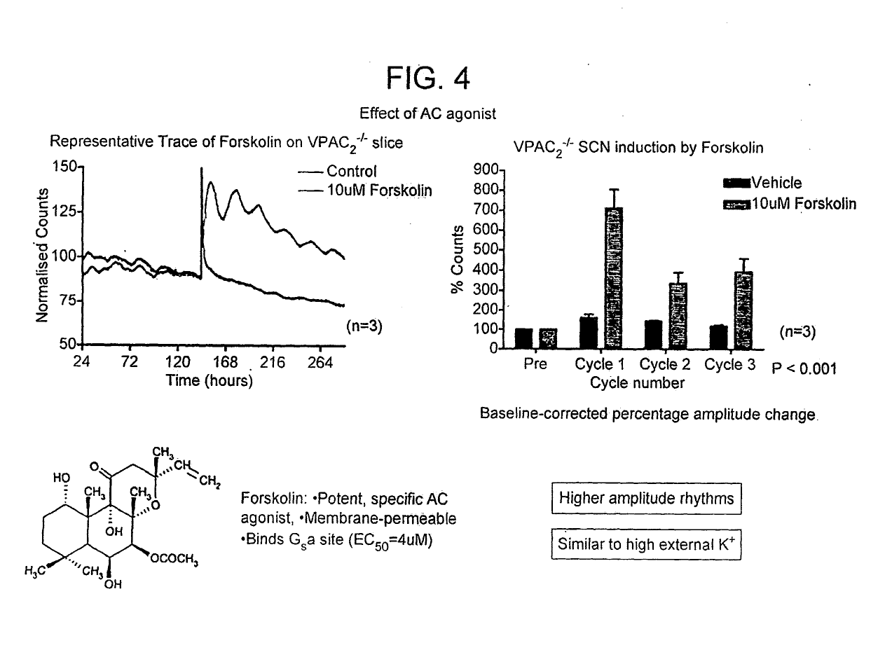

FIG. 4 shows a graph and a bar chart of the effect of AC agonist.

Application of Forskolin restored some activity to VPAC slices.

The appearance is of mimicking the effects of high potassium, and implying greater synchrony between neurons. However, as with potassium treatment, the stronger rhythms do gradually deteriorate. This presumably reflects the upregulation of intracellular PDEs, as rather than restore the proposed circadian increase in cAMP, the treatment has made it constuitively higher.

However, the initial first few cycles following application are unambiguously closer to wild type rhythms, supporting the model disclosed herein.

### Adenylyl Cyclase Inhibitor

In mammals, there are at least ten distinct adenylyl cyclase isozymes, all but one of which are membrane-bound and are central to one of the most important transmembrane signal transduction pathways. The soluble form is regulated by bicarbonate, whereas membrane-bound forms are regulated by numerous neurotransmitters and hormones through cell surface receptors linked via heterotrimeric (αβγ) stimiulatory (Gs) and inhibitory (Gi) guanine nucleotide-dependent regulatory proteins (G-proteins). Most isozymes are activated by Gαs, but differ more significantly in their regulation by Gαi and in the effects of Gβγ. These adenylyl cyclase isozymes exhibit a putative topology with 12 membrane-spanning regions and two ˜40 kDa cytosolic domains (C1 and C2), one after each six membrane-spanning region. C1 and C2 share large conserved regions that interact to form a cleft forming the catalytic active site. N-terminus domains are highly variable and serve regulatory roles. Activation by Gαs occurs through its interaction with the C2 domain of adenylyl cyclase yielding the active enzyme: GTPαsC. Inhibition by G proteins may occur by a direct effect of Gαi with the C1 domain of adenylyl cyclase or by the recombination of βγwith Gαs.

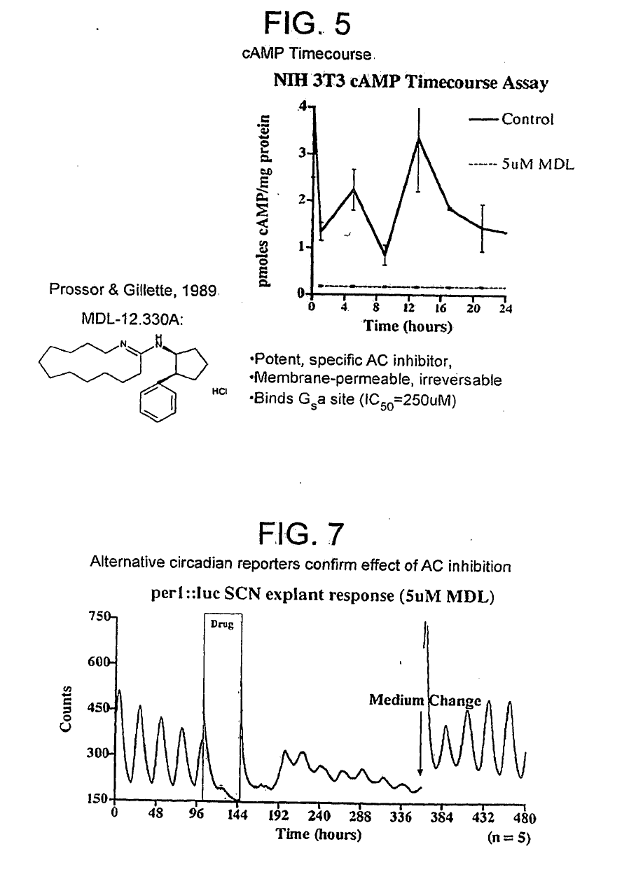

FIG. 5 shows a graph of cAMP timecourse. This experiment is in 3T3 cells. The effect can be seen in the figure.

MDL 12,330A is a potent, specific adenylyl cyclase inhibitor which is membrane permeable and irreversibly binds the Gsα site with an IC₅₀ of 250 um.

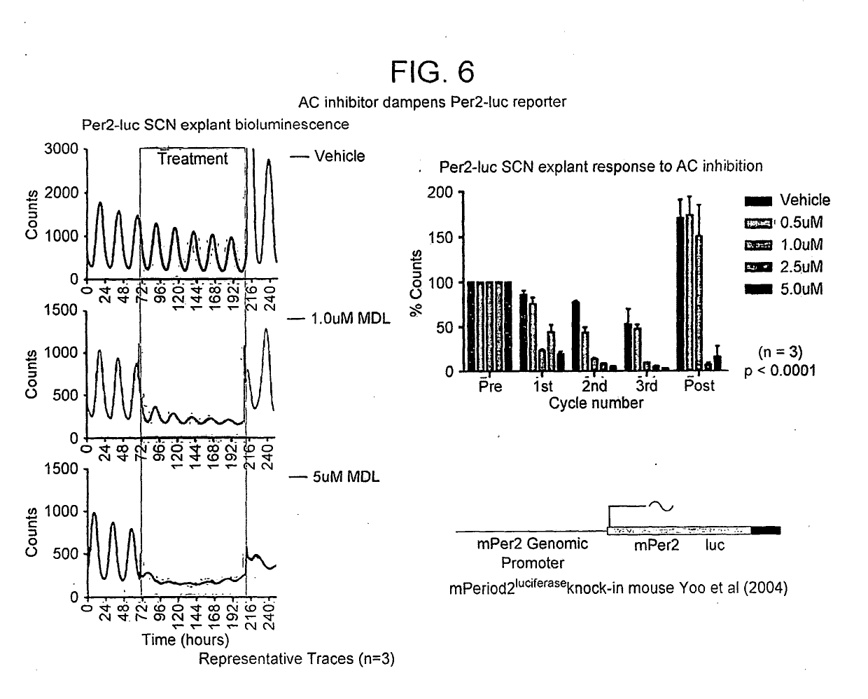

FIG. 6 shows that AC inhibition dampens the Per2-luc reporter.

FIG. 7 shows a graph demonstrating that alternative circadian reporters confirm effect of AC inhibition.

This effect is not caused by toxicity.

This effect was then tested using other reporters such as per1::luc, and bma1::luc.

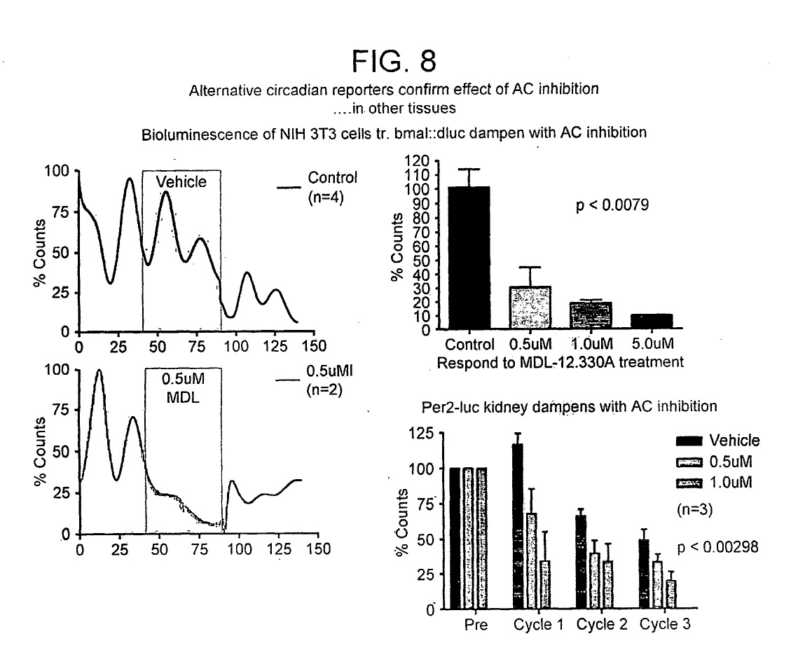

FIG. 8 shows graphs in bar charts of further alternative circadian reporters confirming effect of AC inhibition.

In light of the fact that MDL dampened 3T3s using a different promoter, we investigated whether cAMP signalling is a general requirement for circadian rhythms in mammalian tissues.

Without wishing to be bound by theory, in peripheral tissue endogenous generation of rhythmic second messenger signalling may be essential to sustain rhythmicity, and in the SCN endogenous generation of rhythmic second messenger signalling must be reinforced by rhythmic extracellular stimuli to sustain high amplitude rhythms.

Thus we arrived at a modified view which can be summarised as follows:
VPAC2−/− mice have impaired circadian rhythms because there is no Gs-stimulated rise in cAMP.
cAMP rhythm is essential for rhymnicity in the SCN.
cAMP rhythm is essential for rhythmicity in peripheral tissues.
AC agonist has no period effect

### Conclusions

AC inhibition in wt SCN explants phenocopies VPAC2−/−.
Changes in cAMP signalling may constitute a general core mechanism for sustaining mammalian circadian rhythms.

Without wishing to be bound by theory, we propose that a cytosolic clock may be involved with transcriptional rhythms as an output (eg. cyanobacteria).

The prior alt model accounting for circadian rhythms involves a gene expression/protein feedback system with cAMP signalling relegated to a minor role in phase resetting. We present a fundamentally different new view of circadian rhythms in which cAMP plays a far more central role.

### Example 5

### Manipulation of Circadian Rhythm

We show that circadian adenylyl cyclase activation is essential for setting 24 hour period. We show that inhibition has no effect on the amplitude, only on the period of oscillation. Me demonstrate a 30% increase in period to 31 hours. This is the longest ever observed to date in mammalian circadian biology

We demonstrate that adenylyl cyclase is a clinically relevant intracellular target.

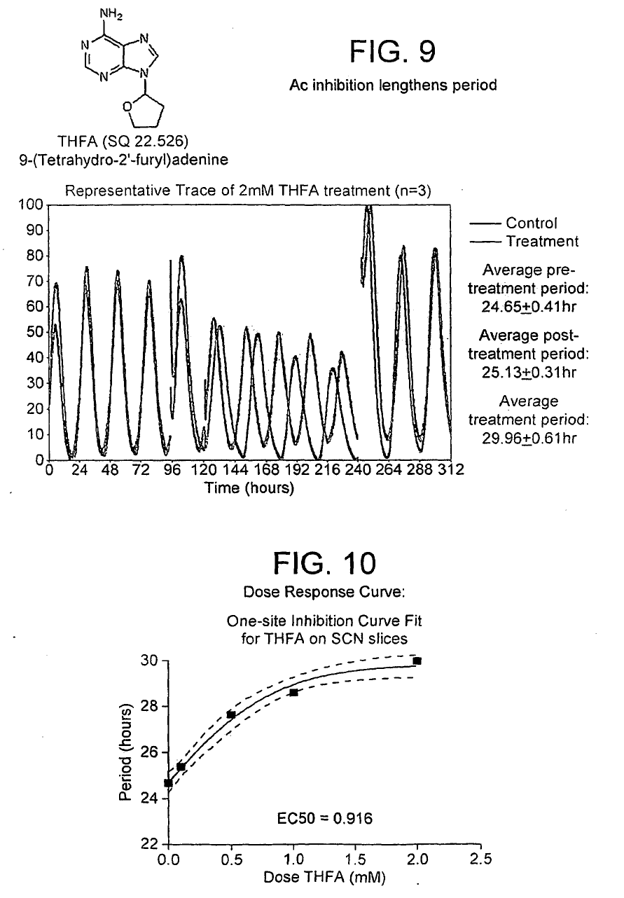

FIG. 9 shows graphs illustrating that adenylyl cyclase inhibition according to the present invention lengthens period.

Even at increased doses there is still no significant amplitude effect.

This is the longest observed mammalian circadian cycle. For comparison, we have studied slices in 10 mM Lithium, which give a 26 hour period.

It should be further noted that washout of the inhibitor restores previous period. This demonstrates the utility of the invention in adjusting circadian rhythm in the short term whilst advantageously avoiding deleterious permanent or long-term extension of the period following a single treatment. In other words, once the subject ceases to take in the active compound in accordance with the invention, their rhythm returns to normal period. Thus, for jet lag, shift lag or related applications then a short-term course of treatment is appropriate to adjust the rhythm which then reverts to approx. 24 hour period. Equally, for FASPS patients, a long-term low-dose daily (once-per-cycle) treatment regime is indicated so that each cycle is slightly lengthened to bring it to approximately 24 hours.

### Example 6

### Dose Response

FIG. 10 shows a dose response curve on SCN slices. The data fit a one-site inhibition model. The data suggest we may be achieving the maximum delay available using this inhibitor.

FIG. 11 shows a dose response curve on 3T3 cells. The data fit a one-site inhibition model: The data suggest we may be achieving the maximum delay available using this inhibitor.

It should be noted that whereas Forskolin (AC agonist) had amplitude effect, THFA (AC inhibitor) has a period effect. This is an extremely surprising and dramatic result.

### Example 7

### Use of P-Site Inhibitor According to the Present Invention

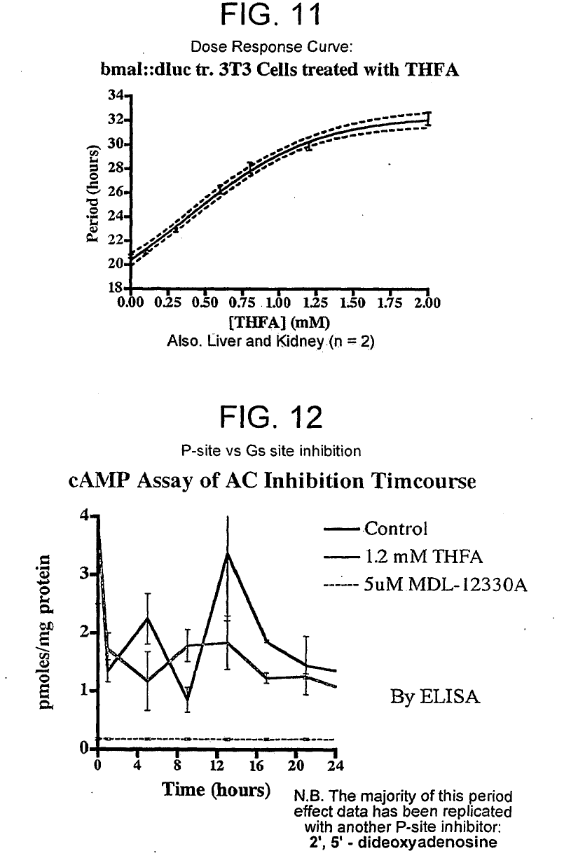

FIG. 12 shows a graph of P-site vs Gs site inhibition. THFA is the P-site inhibitor, MDL is the Gs site inhibitor. P-site inhibitors are preferred according to the present invention.

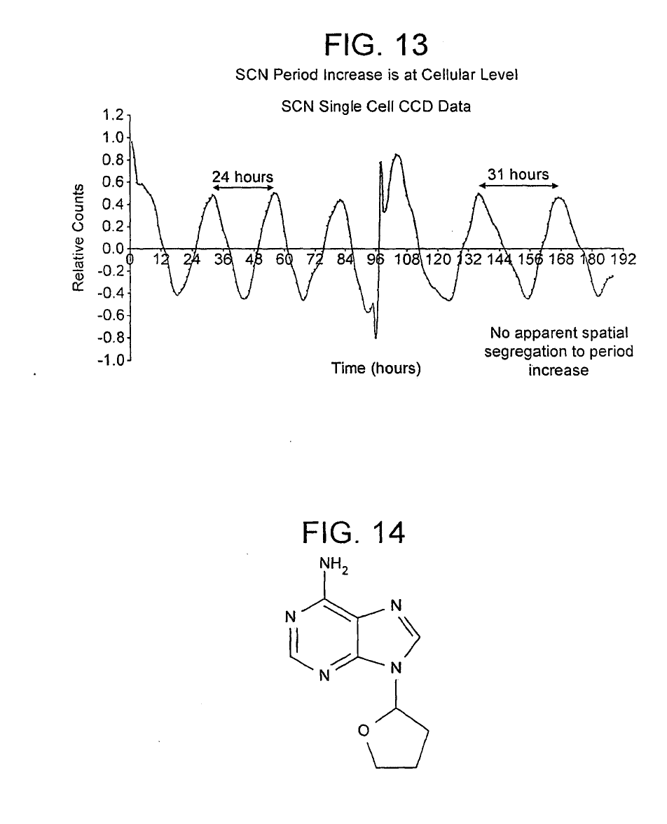

FIG. 13 shows a graph demonstrating that SCN period is at cellular level. This is still true at single neuron level.

We then investigated whether any mechanistic details were implied by the change to the SCN emission waveform.

### Summary

Thus we demonstrate an important clock mechanism, and show how the system can be reliably and reproducibly perturbed by extension of the period of the clock further than ever before. This is useful in the treatment of jet lag, shift lag, and related circadian tai disorders as well as hereditary conditions such as FASPS.

We shows that cAMP signaling constitutes a new level of circadian regulation.

We show that circadian adethylyl cyclase activation is essential for setting 24 hour period.

We show that adenylyl cyclase inhibition leads to unprecedented period lengthening of at least 30-31 hour periods. Inhibition has no effect on amplitude, only period.

We demonstrate the utlity of adenyl cyclase inhibitors, preferably P-site adenylyl cyclase inhibitors, in the treatment of disease-associated with the circadian rhythm.

Thus the importance of cAMP as an intracellular target in alteration of circadian rhythm is demonstrated.

### Example 8

### Manipulation of Circadian Rhythm in Mammals

We demonstrate effects of P-site adenylyl cyclase inhibition in mice by administration of P-site adenylyl cyclase inhibitor. In this example the P-site adenylyl cyclase inhibitor is THFA. FIG. 14 shows the structure of SQ 22536 (9-(Tetrahydro-2′-furyl)adenine (THFA)) which has a Mw of 205.2. In this example the mammals are mice.

### Background

Iwahana et al (Eur J Neurosci. 2004) administered to mice food pellets containing 0.3%. LiCO₃=41 n millimoles lithium/g food. (1 mM lithium final concentration in human serum is non-toxic.)

Matsumoto et al (Learning and memory, 2005) injected mice with 1 mM THFA (=SQ22,536) in 3 ul saline 0.1% DMSO. No significant toxicity is reported.

Simchowitz et al (J Cyclic Nucleotide Protein Phosphor Res. 1983) report that in intact human neutrophils, SQ 22,536 (=THFA) is a non-toxic inhibitor of adenylyl cyclase.

### Experimental

For comparison, slices in 10 mM LiCl₂ appear perfectly healthy and display a 26 hour period.

Slices/3T3 cells/other tissues in 2 mM THFA according to the present invention appear perfectly healthy and display a 30-31 hour period.

Mice used herein typically show adult body weight: 20-40 g, food consumption: 15 g/100 g/day and water consumption: 15 ml/100 g/day.

We assume water intake approximately 5 ml/day.

We assume food intake approximately 5 g/day.

Therefore we treat the mice according to either:

Food intake of 20 micromoles THFA/day=25 mg in 30 g food

or

Water intake of 20 micromoles THFA/day=4 mM solution (add 30 ml water to 25 mg)

Calculations based on the premise that after one day THFA leaves the body at the same rate it enters, infer that serum concentration reaches about 0.6-0.9 mM within a day or so.

Effects on the period of the circadian rhythm are monitored.

### Example 9

### Adenylyl Cyclase Involvement

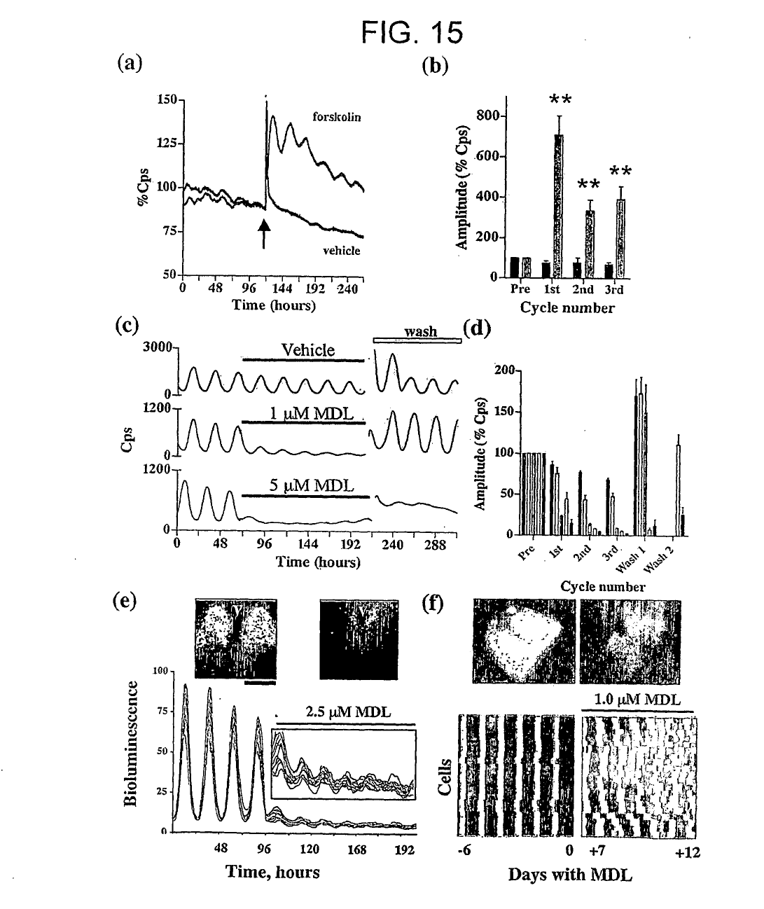

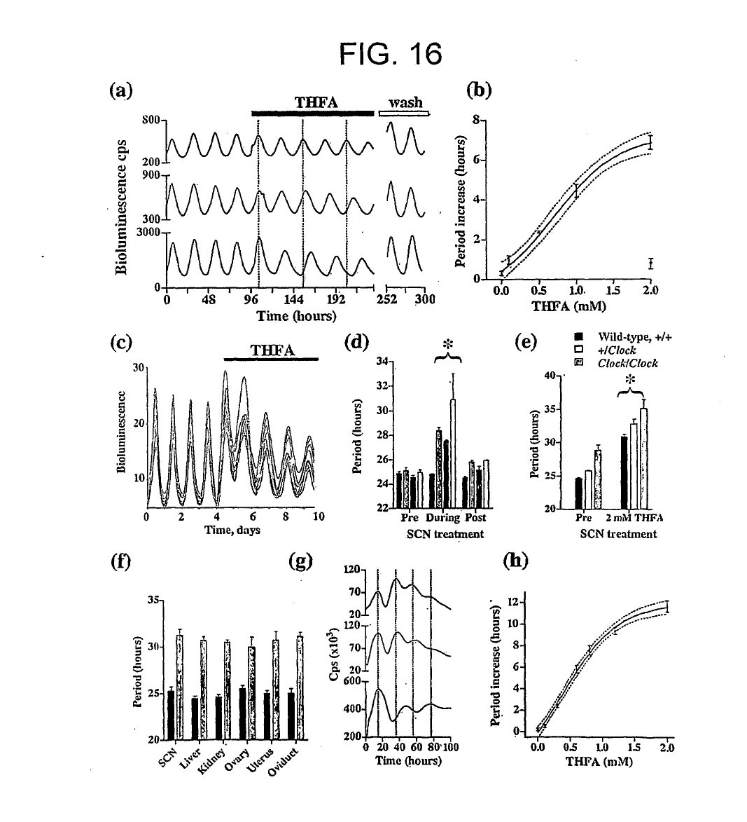

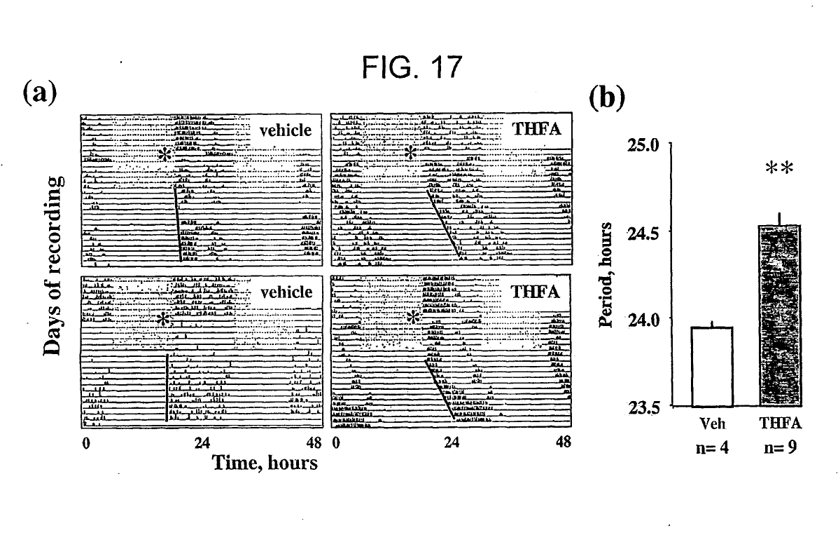

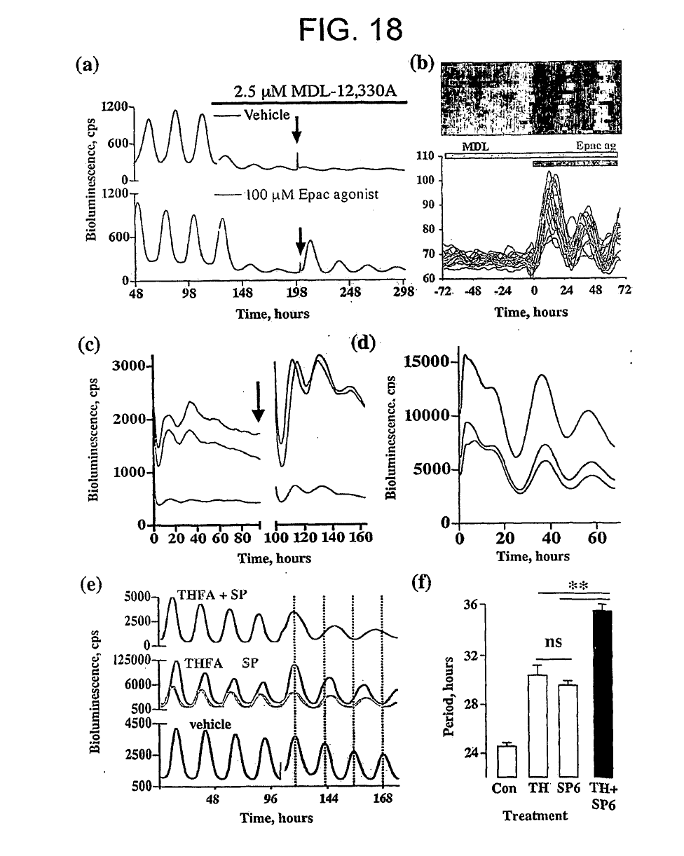

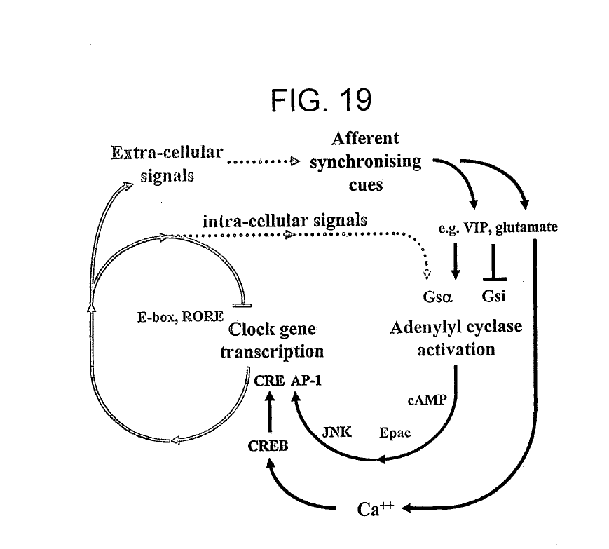

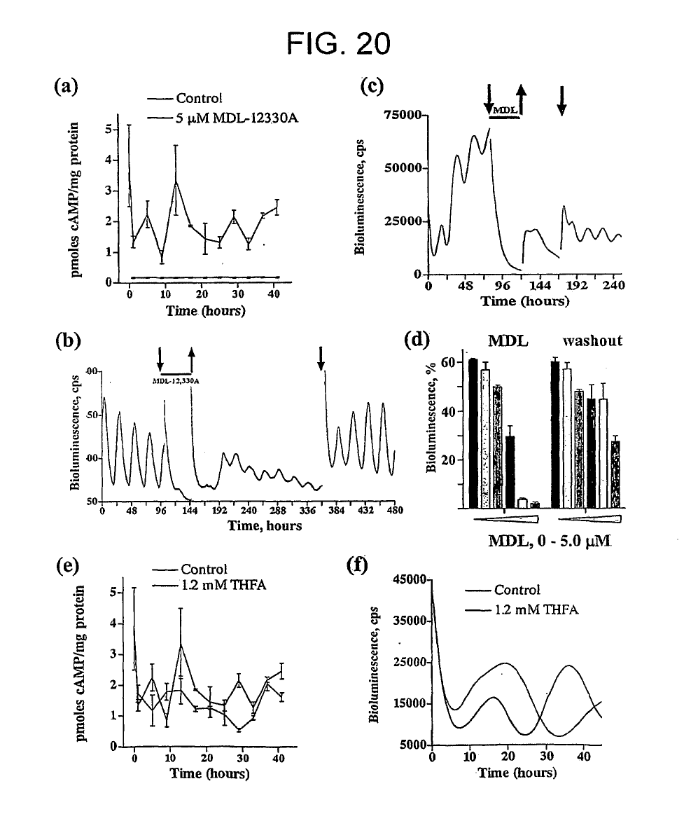

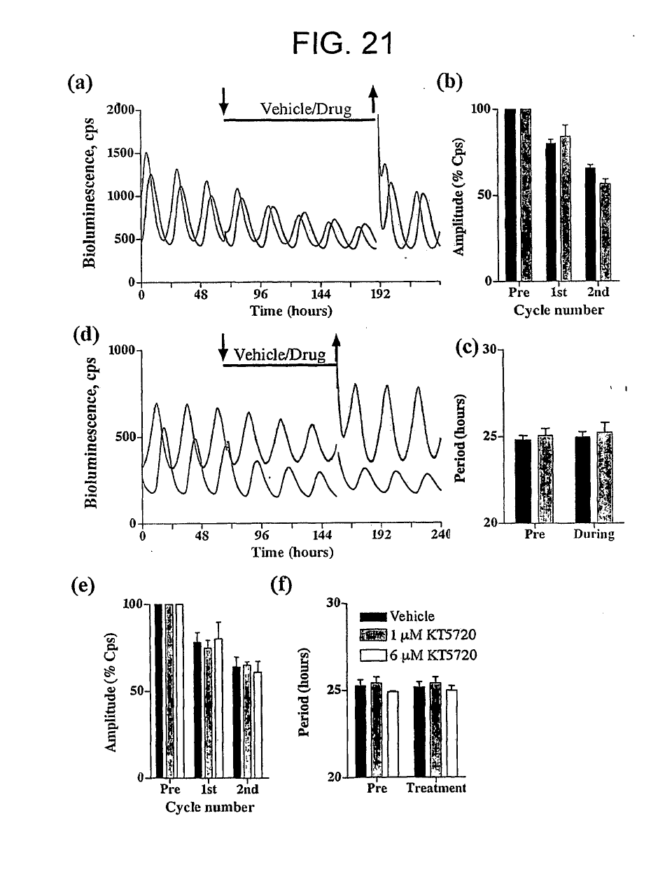

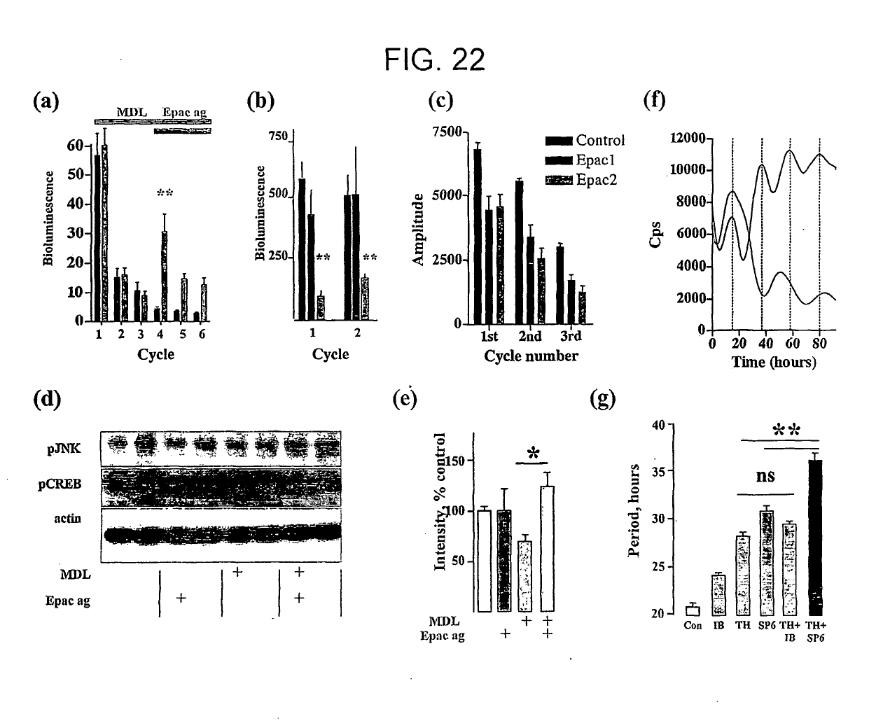

We investigated the possibility that intracellular signalling might sustain the mammalian clock. Buffering of [Ca²⁺]ᵢ attenuates SCN circadian gene expression, and cellular rhythms are both attenuated and desynchronised in the SCN of mice lacking the VPAC2 receptor (Vip2r^{-/-}) for vasoactive intestinal peptide. This receptor is positively coupled to AC and in organotypical Vip2r^{-/-} SCN slices, carrying a mPer1::luciferase reporter, direct activation of AC by forskolin enhances circadian transcriptional activity (FIGS. 15a, b). If AC-dependent signals are necessary for normal clock function, then suppression of AC signals in wild-type SCN should attenuate circadian gene expression and lead to cellular desynchrony. More significantly, if AC is part of the cellular clock, then appropriate manipulation of AC should affect circadian period: a canonical property of the oscillator.

The contribution of endogenous AC to transcriptional cycling in wild-type SCN slices was tested by addition of MDI, 12,330A (MDL), a potent, irreversible AC inhibitor at the Gs_{α} site which at the highest doses used suppressed cAMP levels to below the sensitivity of detection (FIG. 20a). MDL caused a rapid, dose-dependent dampening of circadian cycling, observed with both mPERIOD2-LUCIFERASE (mPER2-LUC) fusion protein and mPer1::luc transcriptional reporter (FIG. 15c, FIG. 20b). Circadian period was not affected. The dampening was reversible upon washout, albeit requiring time to recover from high doses, presumably as new AC was synthesised (FIG. 15d, FIG. 20b). Transcriptional dampening from the SCN slice might arise from loss of individual cellular amplitude and/or cellular desynchrony.

Video imaging of cellular circadian mPER2-LUC expression using a CCD camera revealed a very rapid effect of 2.5 μM MDL across the SCN (FIG. 15e). During prolonged (>7 days)-treatment with an intermediate dose or MDL (1.0 μM) it became obvious that SCN cells not only lost circadian amplitude but also became desynchronised, phenocopying the Vip2r^{-/-} SCN (FIG. 15f). The effect of AC-Gs_{α} inhibition on the clockwork was general and not restricted to the SCN. Reversible, dose-dependent suppression of circadian output by MDL was also evident in organotypic kidney slices from mPER-2-LUC mice and NIH 3T3 cells transfected with a Bmal1::luc reporter (Ueda et al 2005 Nat Genet Vol 37 pp 187-92; see FIG. 20c, d). MDL had no effect on luciferase expression from a control, non-circadian promoter transfected into NIH 3T3 cells.

### Materials and Methods

Recordings of luciferase activity from organotypic slices of SCN or peripheral tissues were made from 5-7 day old pups from PER2::LUC and, Per1::LUC mice using either photomultiplier assemblies for whole tissue emission or CCD camera for single SCN cell imaging (Hamamatsu Photonics Ltd, U.K.) as described May Wood et al 2006 Curr. Biol. Vol. 16 pp 599-605. cAMP-ELISA kit was purchased from R&D systems (Wiesbaden, Germany). NIH 3T3 cells were cultured as described (Kume et al 1999 Cell Vol 98 pp 193-205) and transfected using Genejuice (Novagen, San Diego, Calif.). All drugs were purchased from Sigma-Aldrich (Poole, UK) except PKA/Epac-active cAMP analogues (Axxora; Grünberg; Germany) and KT5720 (Calbiochem, Nottingbam, UK). Drugs were solubilized in medium or dimethylsulfoxide (DMSO) to make stock solutions 50-1000× the working solutions. [DMSO] was never greater than 0.4%. RNA-interference against Epac1 and Epac 2 used pre-validated sequences as instructed (Validated Stealth RNAi, Invitrogen, CA). Western Blots were performed as Reddy et al (2006 Curr. Biol. Vol 16 oo 1107-15). Behavioural rhythms, recorded by running wheel and passive infra-red movement detectors, were analysed in Clocklab (Actimetrics, Evanston Ill.). Central cannulae directed stereotaxically at the SCN and sub-cutaneous minipumps (Alzet® model 1002 pump and brain infusion kit II) for drug infusion were fitted as instructed (Durect Corp. Cupertino, Calif.).

Statistical analysis was performed using Prism GraphPad™ and Statview™. Periods were calculated over ≧3 days recoding For comparative purposes, transcriptional amplitude from PMT recordings was calculated as 100× (post-treatment emission peak-trough/pre-treatment peak-trough). In all cases error bars indicate SEM about the mean where n≧3.

### Example 10

### P-site Inhibition

Loss of SCN circadian amplitude and cellular synchrony in the absence of functional AC does not necessarily establish a role for AC/cAMP signalling in the core oscillator because both may arise from defective clock output. We therefore tested the effect of an alternative form of AC inhibition by using 9-(tetrahydro-2-furyl)-adenine (THFA), a non-competitive AC inhibitor at the purinergic site (p-site). In contrast to MDL, THFA did not alter basal levels of cAMP in fibroblast cultures, but slowed the rate of cAMP synthesis and thereby attenuated peak levels in fibroblasts (FIG. 20e). In mPer1::luc and mPER2-LUC SCN slices THFA caused a robust and dose-dependent increase of circadian period, from ca. 24 to 31 hours (FIGS. 16a, b, c), with some dampening of transcriptional amplitude at higher concentrations. The dose-response was consistent with a one-site inhibition model, saturating at around 2 mM, and was rapidly reversible upon drug washout. Moreover, CCD imaging revealed that THFA increased period in individual neurons across the SCN (FIG. 16c).

The circadian effect of p-site inhibition was confirmed with additional non-competitive inhibitors: 2′5′-dideoxyadenosine and 9-cyclopentyl adenine (FIG. 16d). Period was lengthened in both m Per1:luc and PER2::LUC SCN slices. The effect of THFA on SCN period was additive to that of the Clock mutation (FIG. 16e), suggesting THFA acts in addition to, and independently of, E-box mediated trans-activation by CLOCK. Importantly, 2 mM THFA also caused a dramatic increase in circadian period in all peripheral tissues tested from mPER2-LUC mice (FIG. 16f), whilst in fibroblasts transfected with mBmal::luc reporter there was an even more pronounced effect, circadian period lengthening from ca. 21 to 31 hours (FIGS. 16g, h). Inhibition of AC also lengthened circadian period of wheel-running behaviour when delivered to the SCN of centrally cannulated mice. Following recovery from surgery, the activity/rest cycles of vehicle-treated mice were clearly entrained to the light-dark cycle, activity onset coinciding with lights off, and upon release into continuous dim red light they free-ran with a period very close to 24 hours (FIGS. 17a, b). In contrast, in all nine mice that received chronic central THFA, free-running circadian period was significantly (p<0:01, t-test) lengthened upon transfer to continuous dim red light.

### Example 11

### JNK Inhibition

Protein kinase A (PIA) is a candidate for mediating circadian effects of cAMP, so mPer1::luc and mPER2-LUC SCN slices were incubated with a series of well characterized PKA inhibitors targeted against either the regulatory or the catalytic sub-unit. Surprisingly, there was no significant effect on either circadian amplitude or period (FIG. 21).

Epac1/2 are reported to be alternative mediators of AC signalling. To test their putative role mPer1:Lluc SCN slices were incubated with Sp-8-CPT-2′-O-Me-cAMPS, a specific Epac agonist. This had no significant effect on rhythmic transcriptional amplitude or period. When mPER2-LUC SCN slices were treated with 2.5 μM MDL to induce dampening, however, the Epac-specific agonist dramatically restored circadian output, higher amplitude rhythms persisting for several cycles (FIG. 18a, FIG. 22a); thereby showing that activation of downstream Epac activity can compensate for the effect of AC inhibition of circadian gene expression; CCD recording revealed that Epac agonist transiently activated and re-synchronised circadian gene expression in MDL-treated SCN neurons (FIG. 18b). Further, when fibroblasts carrying the Bmal1::luc reporter were transfected with a known inhibitor of Epac, HA-Rap1(S17N), circadian gene expression was markedly dampened (FIG. 18c, FIG. 22b). Wild-type Rap1, which is not effective at Epac had no effect. Furthermore, RTAi knock-down of endogenous Epac1 and Epac 2 in fibroblasts similarly dampened circadian gene expression (FIG. 18d, FIG. 22c). Epac is reported to activate c-Jun N-terminal kinase (JNK) p46, and pJNK in turn activates gene expression through transcription factors of the AP-1 family. Given that Period genes contain AP-1 regulatory sequences, we conceived a model whereby cyclical activation of cAMP/Epac/JNK/AP-1 drives circadian pacemaking. Consistent with this model, Epac agonist increased pJNK (but not pCREB) fibroblasts given 5 μM MDL (FIGS. 22d, e), and JNK inhibition lengthened circadian period in SCN slices and fibroblasts comparable to the effect of AC inhibition (FIGS. 18e, f, FIGS. 22f, g). Importantly, the effect of JNK inhibition was additive to that of THFA, leading to unprecedented lengthening of the SCN and fibroblast clockwork to around 36 hours. In contrast, IBMX which lengthened period in fibroblasts did not enhance the effect of THFA, presumably because both act via perturbing cAMP levels and so have redundant effects (FIG. 22g).

### Summary

Our results demonstrate that AC activity, likely mediated via Epac/JNK, determines the amplitude and period of circadian transcriptional loops, and that interference with AC signalling desynchronizes SCN clock cells, probably because loss of circadian amplitude compromises inter-neuronal signals, such as VIP release, that constitute an output from the core oscillator. We therefore propose that circadian timing in mammals is sustained and its period determined by a reciprocal interplay in which transcriptional/post-translational feedback loops and neural activity drive intracellular rhythms of AC signalling. This cytosolic-rhythm in turn potentiates transcriptional cycles i.e. clock output constitutes an input into the current and/or subsequent cycle (FIG. 19). The dependence of transcriptional cycles on cAMP signalling explains the compromised circadian gene expression and behaviour in Vip2r^{-/-} mice, and is likely applicable to local clockworks in major organs where afferent signals other than VIP regulate AC activity. It may also extend to the Drosophila clock, which is maintained by the PDF receptor (a homologue of VPAC2 receptor) that activates AC. The differential effect of AC inhibitors (Gₛα vs. p-site) likely reflects their particular actions on AC kinetics, and non-competitive p-site inhibitors such as THFA may be of broad therapeutic application where either acute (jet-lag, shiftwork) or maintained (Familial Advanced Sleep Phase Syndrome) extension of circadian period in all body tissues is indicated.

All publications mentioned in the above specification are herein incorporated by reference. Various modifications and variations of the described methods and system of the present invention will be apparent to those skilled in the art without departing from the scope of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

1. (canceled)

2. A method of extending the period of circadian rhythm in a subject, said method comprising administering to said subject a composition comprising an inhibitor of adenylyl cyclase, wherein said inhibitor of adenylyl cyclase is a purine site (p-site) inhibitor.

3. A method of modifying the circadian rhythm in a subject comprising administering to said subject a composition comprising an inhibitor of adenylyl cyclase, wherein said inhibitor of adenylyl cyclase is a purine site (p-site) inhibitor.

4. The method of claim 3, wherein said subject is suffering from or at risk of suffering from jet lag.

5. The method of claim 3, wherein said subject is suffering from familial advanced sleep phase syndrome.

6. The method of claim 3, wherein said subject is suffering from or at risk of suffering from shift lag.

7. The method of any one of claims 2 or 3, wherein said inhibitor of adenylyl cyclase is a purine site ligand.

8. The method of any one of claims 2 or 3, wherein said inhibitor of adenylyl cyclase is specific for adenylyl cyclases.

9. The method of any one of claims 2 or 3, wherein said inhibitor of adenylyl cyclase acts at the P-site of adenylyl cyclase.

10. The method of any one of claims 2 or 3, wherein said inhibitor of adenylyl cyclase is selected from the group consisting of 9-(tetrahydrofuryl)-adenine, 9-(cyclopentyl) adenine and 2′,5′-dideoxyadenosine.

11. The method of any one of claims 2 or 3, wherein said inhibitor of adenylyl cyclase is 9-(tetrahydrofuryl)-adenine.

12. The method of any one of claims 2 or 3, wherein said composition further comprises a c-Jun N-terminal kinase (JNK) inhibitor.

13. The method according to claim 12 wherein said JNK inhibitor is SP600125 (anthra[1,9-cd]pyrazol-6(2H)-one; 1,9-pyrazoloanthrone; SAPK inhibitor II).

14-24. (canceled)

25. A pharmaceutical pack or kit comprising
(i) an adenylyl cyclase inhibitor, and
(ii) a JNK-inhibitor.

26. A pharmaceutical pack or kit according to claim 25 wherein said adenylyl cyclase inhibitor is 9-(tetrahydrofuryl)-adenine.

27. A pharmaceutical pack or kit according to claim 25 or 26 wherein said JNK inhibitor is SP600125 (anthra[1,9-cd]pyrazol-6(2H)-one; 1,9-pyrazoloanthrone; SAPK inhibitor II).
